(19) United States
(12) Patent Application Publication
(10) Pub. No.: US 2023/0349893 A1
(43) Pub. Date: Nov. 2, 2023

(54) LUBRICANT-INFUSED SURFACE BIOSENSING INTERFACE, METHODS OF MAKING AND USES THEREOF

(71) Applicant: McMaster University, Hamilton (CA)

(72) Inventors: Tohid Didar, Dundas (CA); Amid Shakeri, Hamilton (CA); Hanie Yousefi, Toronto (CA)

(21) Appl. No.: 18/027,587

(22) PCT Filed: Sep. 22, 2021
(86) PCT No.: PCT/CA2021/051320

§ 371 Date: Mar. 21, 2023

Related U.S. Application Data
(60) Provisional application No. 63/081,622, filed on Sep. 22, 2020.

(51) Int. Cl. G01N 33/543

(52) U.S. Cl. CPC G01N 33/54353

(57) ABSTRACT

This application relates to a method for fabricating a biofunctionalized surface on a substrate, wherein the substrate comprises hydroxyl groups on the surface to be biofunctionalized, the method comprising: covalently attaching organosilane groups to less than all of the hydroxyl groups on the surface of the substrate; covalently attaching one or more biospecies to the surface of the substrate; and applying a lubricant to the substrate, wherein the biospecies comprises a biorecognition element that detects a target analyte in a sample. A biofunctionalized surface made therefrom and use thereof, such as for biosensing applications, are also disclosed.

### CROSS-REFERENCE TO RELATED APPLICATION

The present application claims priority to co-pending U.S. provisional patent application No. 63/081,622, which was filed on Sep. 22, 2020, the contents of which are incorporated herein by reference in their entirety.

### FIELD

The present disclosure generally relates to biofunctional surfaces, and in particular, methods to covalently micro/nano pattern such surfaces and their application in biosensors and biomedical assays.

### BACKGROUND

Biofunctional interfaces capable of selectively anchoring biomolecules of interest onto a platform are the key components of many biomedical assays, clinical pathologies, and medical implants [1,2]. Biosensor industries, as a prime example, are progressively looking for innovative approaches to modify the surface functionalization process for enhancing the limit of detection of sensors used in healthcare monitoring, on-chip screening for disease and point-of-care diagnostic devices. In addition, proper design of interfaces coated with biomolecules, cells, viruses and nano-coatings would result in developing interfaces with superior capabilities for drug delivery, anti-fouling, anti-chromogenicity, self-cleaning as well as evaluating and eliminating pollutants in environmental and agricultural applications [3-5]. Furthermore, in cellular and biochemical assays where cellular processes are detected and quantified, proper surface functionalization allows investigators to precisely control protein binding and guide cell growth [6,7].

Biofunctional surfaces, or more generically functional surfaces, could also be used to covalently bond micro/nano particles as well as other functional entities with the substrates [8,9]. TiO₂ nanoparticles, for example, have great photocatalytic properties which are widely employed in environmental and purification applications [10]. Combining a strong and durable TiO₂ coating on different substrates with entities (nanoparticles, biomolecules, viruses, cells, etc.) that provide functionality, such as specificity to the target biospecies, would be useful for robust biosensors as biomedical assays and diagnostics.

Human interleukin-6 (IL-6) is a multifunctional, pro-inflammatory cytokine that has been found to be overexpressed in viral infections, inflammatory conditions and several cancer types such as lung, colorectal, breast, and prostate cancers [11-17] as well as in respiratory infections caused by Severe Acute Respiratory Syndrome Corona Virus 2 (SARS-CoV-2). [18-20] The level of expression in plasma typically reflects severity of the disease, where significantly elevated levels indicate aggressive tumor growth or viral load and poor prognosis in patients.[12,20] Additionally, IL-6 is an important anti-inflammatory cytokine that induces acute responses in chronic inflammatory pathologies. As such, there has been an increasing interest in the use of IL-6 as a biomarker for the diagnosis of early stages of viral infections, cancer, and chronic inflammation. [19-21] A practical IL-6 biosensor should provide a low limit of detection (LOD) (≤5 pg mL⁻¹) and acceptable linear dynamic range (1 pg mL⁻¹ to 100 pg mL⁻¹) in complex fluids, in addition to accuracy, facile operation, and amenable to mass production. [21,22]

There are a large number of different IL-6 detection techniques that have been reported in the literature including electrochemical sensors,[23-32] surface plasmon resonance (SPR),[33-35] chemiluminescence immunoassay (CLIA),[36-39] and immunofluorescence assays (IFA),[40-43] Utilizing zero- and one-dimensional materials such as carbon nanotubes (CNTs), [24,26] nanoparticles and nanowires,[23,29] as well as porous nanoparticles,[15] optical fibers,[42] and microfluidic platforms,[38] have enabled higher sensitivity in IL-6 detection and to date, electrochemical methods have proven to be the most promising candidate for detection of IL-6 at very low concentrations (0.33 pg mL⁻¹ in buffer) with a wide linear dynamic range. [32] While reported IL-6 biosensors have demonstrated satisfactory LODs in buffer or processed serum, their performance in human whole plasma declines significantly, leading to higher LOD's and/or false positive results. In electrochemical sensors, for example, the non-specific attachment of biological entities in plasma or blood can interfere with the resistivity at the electrodes thereby deteriorating their sensitivity for detection of IL-6 at clinically relevant concentrations.[44] So far, the lowest theoretical LOD for IL-6 detection in plasma was reported by Sabaté del Rio et al.[23] This method utilized a complex system composed of 3D BSA nanocomposite, CNTs/Au-nanoparticles, and Au-nanowires and electrochemical detection to obtain an LOD of 23 pg mL⁻¹ in human plasma, which exceeds the typical sensitivity requirements of <5 pg mL⁻¹.

Precise patterning of a surface with the desired biomolecule is of considerable importance for selective screening in biosensors and biological assays. Micro/nano printing methods could provide access to separated bio-functional areas in order to investigate the status of multianalyte in high throughput systems. Moreover, in tissue engineering, it is required to position biomolecules in distinct locations to promote cell attachment on those areas, while preventing cell attachment on undesired parts [45]. Microcontact printing method is one of the most widely-used technique to form various patterns on surfaces [46,47]. One major problem with this technique is physical attachment of biomolecules to the surface. As a result, the created patterns cannot resist harsh in vivo and in vitro environments where the high shear stress, for instant, can lead to detachment of the biomolecule from the surface. Microcontact printing of (3-Aminopropyl)triethoxysilane (APTES) on an plasma activated surfaces can be an alternative way to create a covalent bond between the amine terminated groups of the surface and carboxylic groups of the target biospecies [48]. The procedure, however, is fairly challenging and time consuming.

Seeking the most durable and appropriate blocking agent is the other decisive factor drawing a lot of attention in biosensing, bio-chemical assays, implants and other functional substrates. Poly(ethylene glycol) (PEG), poly(acrylamide)s, poly(N-vinylpyrrolidone), bovine serum albumin (BSA), milk powder, and Tween™ 20 are some of the common blocking agents used to prevent non-specific binding [49-51]. Although these blocking agents have been widely used on biofunctional surfaces and could block the surface to a great extent, there are some drawbacks associated with them. For example, one of the disadvantages of the blocking agents such as PEG, poly(acrylamide)s, and poly(N-vinylpyrrolidone) is inevitable formation of defects in the surface chemistry which leads to biomolecules attachment and biofouling [52]. Moreover, it has been shown that BSA, milk, and Tween 20 can sometimes disturb the sensitivity of the assays by interfering with immunochemical reactions or incomplete saturation [53-55].

Omniphobic lubricant-infused surfaces (LISs),[56] have aroused interest as anti-biofouling coatings in recent years due to their ability to repel bacteria, blood cells, proteins, as well as their non-wetting properties to different fluids. [57-60] This property is caused by a slippery or low surface tension interface between a monolayer of lubricant, locked into a porous or rough surface and the biofluid or immiscible liquid to be repelled.[61] The omniphobic LIS technology has been employed for antibacterial applications, as well as medical implants and devices where thrombosis and infections could pose a threat;[62-65] however, they have not been implemented as blocking agents for biosensing.

The background herein is included solely to explain the context of the application. This is not to be taken as an admission that any of the material referred to was published, known, or part of the common general knowledge as of the priority date.

### SUMMARY

The present application includes a method for fabricating a biofunctionalized surface on a substrate, wherein the substrate comprises hydroxyl groups on the surface to be biofunctionalized, the method comprising: covalently attaching organosilane groups to less than all of the hydroxyl groups on the surface of the substrate; covalently attaching one or more biospecies to the surface of the substrate; and applying a lubricant to the substrate, wherein the biospecies comprises a biorecognition element that detects a target analyte in a sample.

In accordance with an aspect, there is provided a biofunctionalized surface comprising a substrate functionalized with a silane and a covalently-bound biospecies, wherein the biospecies comprises a biorecognition element capable of detecting a target analyte in a sample.

In some embodiments, the substrate comprises a metallic, polymeric and/or glass material, optionally a nanoparticle.

In some embodiments, the silane comprises a fluorosilane.

In some embodiments, the fluorosilane comprises 1H,1H,2H,2H-perfluorooctyltriethoxysilane, 2-(perfluorodecyl)ethyl acrylate, 1H,1H,2H,2H-perfluorodecanethiol, trichloro(1H,1H,2H,2H-perfluorooctyl)silane and/or 1H,1H,2H,2H-perfluorodecyltrimethoxysilane.

In some embodiments, the silane comprises n-propyltrichlorosilane.

In some embodiments, the biofunctionalized surface further comprises micro- or nano-sized structures on the surface.

In some embodiments, the biofunctionalized surface further comprises a lubricant.

In some embodiments, the lubricant comprises a perfluorotrialkylamine, a perfluoroalkylether or perfluoroalkylpolyether, a perfluoroalkane, a perfluorocycloalkane and/or a perfluorohaloalkane.

In some embodiments, the biospecies is functionalized with a covalent crosslinking agent.

In some embodiments, the covalent crosslinking agent comprises a silane coupling agent.

In some embodiments, the silane coupling agent comprises a mono-, di- or tri-functional silane.

In some embodiments, the silane couple agent is selected from (3-aminopropyl)triethoxysilane (APTES), (3-aminopropyl)trimethoxysilane (APTMS), 3-mercaptopropyl trimethoxysilane (MPTMS) and/or glycidyloxypropyl)trimethoxysilane (GLYMO).

In some embodiments, the crosslinking agent comprises a carbodiimide crosslinker, glutaraldehyde and/or succinimide ester.

In some embodiments, the covalent crosslinking agent comprises a polymer, optionally in combination with a silane.

In some embodiments, the polymer comprises cyclophane-containing polymers, poly(allylamine hydrochloride), hexamethylenediamine, 1,3-diaminopropane, poly(ethyleneimine), poly(acrylic acid), functional polyethylene glycol (PEG) (e.g. NHS-PEG), amine functional polyacrylamide, and/or hyperbranched polyglycerol.

In some embodiments, the biospecies comprises a biomolecule, virus, cell and/or tissue.

In some embodiments, the biomolecule comprises a protein, peptide and/or nucleic acid.

In some embodiments, the biospecies further comprises a nanoparticle.

In some embodiments, the biospecies are positioned in a distinct pattern on the surface.

In accordance with another aspect, there is provided a biosensor comprising the biofunctionalized surface disclosed herein.

In some embodiments, the biofunctionalized surface is capable of preventing non-specific adsorption.

In some embodiments, the biosensor provides and multiplex detection of different target analytes.

In some embodiments, the biosensor is used for clinical and agricultural diagnostics, agri-food quality control, environmental monitoring, health screening, health monitoring, and/or pharmaceutical development.

In accordance with another aspect, there is provided a device comprising the biofunctionalized surface disclosed herein.

In accordance with another aspect, there is provided a device comprising the biosensor disclosed herein.

In accordance with another aspect, there is provided a method for fabricating the biofunctionalized surface, the method comprising hydroxylating the substrate, silanating the substrate, covalently attaching a biospecies onto the substrate, and optionally applying a lubricant onto the substrate, wherein the biospecies comprises a biorecognition element capable of detecting a target analyte in a sample.

In some embodiments, hydroxylating the substrate comprises plasma treatment, piranha etching, Ultraviolet/Ozone treatment and/or corona discharge.

In some embodiments, plasma treatment comprises using gaseous air, O₂, CO₂ or a combination thereof.

In some embodiments, silanating the substrate comprises chemical vapor deposition or liquid phase deposition.

In some embodiments, silanating the substrate comprises deposition of a fluorosilane.

In some embodiments, the method further comprises hydroxylating the surface after silanating the substrate.

In some embodiments, the method further comprises plasma treatment after silanating the substrate.

In some embodiments, plasma treatment comprises gaseous air, O₂, CO₂, allylamine plasma, ammonia plasma, and/or nitrogen plasma.

In some embodiments, covalently attaching a biospecies comprises applying a covalent crosslinker to the substrate before applying the biospecies to the substrate.

In some embodiments, covalently attaching a biospecies comprises combining a covalent crosslinker with the biospecies into a mixture then applying the mixture to the substrate.

In some embodiments, covalently attaching a biospecies comprises positioning the biospecies in a distinct pattern on the surface.

In some embodiments, covalently attaching a biospecies comprises non-contact printing, optionally inkjet printing and/or spraying.

In some embodiments, covalently attaching a biospecies comprises contact printing, optionally microcontact printing, roll-to-roll printing and/or stamping.

In accordance with another aspect, there is provided use of the biofunctionalized surface disclosed herein.

Other features and advantages of the present application will become apparent from the following detailed description. It should be understood, however, that the detailed description and the specific examples, while indicating embodiments of the application, are given by way of illustration only and the scope of the claims should not be limited by these embodiments, but should be given the broadest interpretation consistent with the description as a whole.

### DRAWINGS

Certain embodiments of the application will now be described in greater detail with reference to the attached drawings in which:

FIG. 1 shows a schematic representation of fabricating repellent functional surfaces in exemplary embodiments of the application, starting with CO₂ plasma treatment and subsequent fluorosilanization process, followed by EDC-NHS activation of the carboxylic groups over the surface which can then bind to the amine groups of the desired functional entities.

FIG. 2 shows fluorescent images of the surfaces micro patterned with EDC-NHS and then incubated with BSA-FITC in exemplary embodiments of the application: (a) Surfaces were CO₂ plasma treated before the silanization process; (b) Surfaces were O₂ plasma treated before the silanization process (scale bars are 200 μm).

FIG. 3 shows fluorescent images of the surfaces micro patterned with EDC-NHS and then incubated with BSA-FITC on surfaces treated with CO₂ plasma before the salinization process (scale bar is 200 μm) in exemplary embodiments of the application.

FIG. 4 shows fluorescent images of the surfaces micro patterned with either EDC (a) or NHS (b) and then incubated with BSA-FITC (scale bars are 200 μm) in exemplary embodiments of the application.

FIG. 5 shows fluorescent images of the surfaces micro patterned with BSA-FITC mixed with EDC-NHS (scale bar is 200 μm) in exemplary embodiments of the application.

FIG. 6 shows fluorescent images of glass (a) and polystyrene (b) surfaces micro patterned with amine conjugated fluorescently labeled DNA mixed with EDC-NHS (scale bar is 200 μm) in exemplary embodiments of the application.

FIG. 7 shows microcontact printing EDC-NHS solution onto the CO₂ plasma treated surface and then the entire surface was incubated with CD34 in exemplary embodiments of the application.

FIG. 8 shows a schematic representation of the process for producing PMMA-based antibody embedded lubricant-infused sensors for IL-6 detection in exemplary embodiments of the application.

FIG. 9 shows the deconvoluted high resolution XPS spectra in exemplary embodiments of the application: (a) C1s; (b) O1s; (c) F1s/O1s peak area ratio before and after PMMA surface modification; data are shown as mean±SD; (d) F1s, for the PMMA surface before plasma treatment (plain), after the plasma treatment (plasma), and after fluorosilanization (FS).

FIG. 10 shows the contact angle and sliding angle results of PMMA surfaces before and after the surface modification in exemplary embodiments of the application (error bars represent the standard deviation; there is a significant difference in the contact angles with *P<<10⁻⁷).

FIG. 11 shows a sandwich assay, representative fluorescence images, the linear dynamic range and blood clots imaged in exemplary embodiments of the application: (a) Schematic representation of FS treated PMMA surface patterned with the capture antibodies; (b) Schematic representation of FS treated PMMA surface after adding the lubricant and performing the IFA. The lubricant layer repels all sorts of biofluids and proteins while capturing the target at the printed areas; (c) and (d), illustrates representative fluorescence raw images of the microarrays of IL-6 at a concentration of 312.5 pg mL⁻¹ before and after the Chan-Vese segmentation; (e) Linear dynamic range of dose response curves of the IL-6 IFA using LIS where graphical data are shown as mean (n=18 replicates, R2 ≥0.97 for buffer and ≥0.98 for plasma) with linear range of 0.5-156 pg mL⁻¹ (error bars represent the ±standard deviation); (f) and (g), Optical microscope images of the blood cells attached to the untreated (plain) PMMA and LIS PMMA, respectively, following whole blood coagulation test. Inset images show the wells after the clotting assay (well area is ˜1 cm²); (h) SEM image of the blood clot attached to the plain PMMA surface; (i) SEM image of antibody-printed LIS PMMA surface after the clotting assay.

FIG. 12 shows the IFA results of microarrays of IL-6 diluted in buffer at different concentrations (scale bar is 100 μm) in an exemplary embodiment of the application.

FIG. 13 shows the IFA results of microarrays of IL-6 diluted in plasma at different concentrations (scale bar is 100 μm) in an exemplary embodiment of the application.

FIG. 14 shows the Chan-Vese image segmentation processing of the LIS IL-6 IFA dose response in pg mL⁻¹ of buffer in an exemplary embodiment of the application.

FIG. 15 shows the dose response curves of the LIS IL-6 IFA in exemplary embodiments of the application: graphical data are shown as mean (n=18 replicates) fitted by a quadratic trendline with goodness of fit R2 ≥0.99 for buffer and ≥0.98 for plasma; reportable range is 1-312.5 pg mL⁻¹; there is a significant difference between the LOD of 0.5 and zero concentration with P<0.0005; in the inset equations, c is the IL-6 concentration in pg mL⁻¹.

FIG. 16 shows the fluorescence microscopy image of IL-6 IFA patterns captured from recalcified citrated blood at the concentration of 312.5 pg mL⁻¹ before and after Chan-Vese image segmentation processing in exemplary embodiments of the application: recovery of the spiked sample (n=9 replicates) on the plasma standard curve was 119.4% indicating the functionality of LIS-IFA for detection of IL-6 in whole blood.

FIG. 17 shows a schematic illustration of LIS-DNAzyme sensors detecting E. coli cells in milk in exemplary embodiments of the application.

FIG. 18 shows TAMRA-labeled DNAzyme cleavage activity in presence of E. coli cells in exemplary embodiments of the application: Clv and Unclv represent the cleavage product and the full-length DNAzyme (which is uncleaved) respectively; cleaved band in presence of E. coli cells is indicated with a box showing modifications applied to the probe did not affect its activity.

FIG. 19 shows a signal-to-noise ratio comparison of various blocking agents in exemplary embodiments of the application: a) fluorescence image of the surface with a Texas red-labeled ssDNA (TRDNA) in milk with (a1) no blocking agent; (a2) PLL-PEG; (a3) BSA; (a4) lubricant; and (a5) fluorescence quantification of (a1), (a2), (a3), and (a4); b) Fluorescence image of the surface with the DNAzyme in milk spiked with E. coli cells, with (b1) no blocking agent; (b2) PLL-PEG; (b3) BSA; (b4) lubricant (LIS-DNAzyme sensor); and (b5) fluorescence intensity quantification of (b1), (b2), (b3), and (b4) (using 10⁶ CFU/mL of E. coli cells).

FIG. 20 shows the importance of epoxy for DNAzyme printing onto the surfaces using fluorescence imaging of the surface with TAMRA-labeled DNAzyme without epoxy before washing (a), and after washing (b) in exemplary embodiments of the application.

FIG. 21 shows (a) fluorescence image of the surface with the printed DNAzyme after incubation in non-contaminated milk; (b) fluorescence image of the surface with the printed DNAzyme after incubation in milk spiked with E. coli cells (with lubricant: LIS-DNAzyme biosensor using 10⁶ CFU/mL of E. coli cells; (c) fluorescence quantification of DNAzyme sensors incubated with contaminated milk with and without LIS coating in exemplary embodiments of the application.

FIG. 22 shows detection of bacterial contamination in milk samples after LIS-DNAzyme biosensors were incubated in milk spiked with different concentrations of E. coli cells for 1 hour at room temperature in exemplary embodiments of the application.

FIG. 23 shows a comparison of the limit of detection of the LIS-DNAzyme and non-LIS sensors in milk spiked with different concentrations of E. coli cells for 1 hour at room temperature in exemplary embodiments of the application.

FIG. 24 shows fluorescence image of BSA-FITC conjugated with GLYMO microcontact printed onto an FS treated PMMA substrate, compared to a control sample where unconjugated BSA-FITC was patterned, in exemplary embodiments of the application.

### DETAILED DESCRIPTION

### I. Definitions

Unless otherwise indicated, the definitions and embodiments described in this and other sections are intended to be applicable to all embodiments and aspects of the present application herein described for which they are suitable as would be understood by a person skilled in the art. It is also to be understood that the terminology used herein is for the purpose of describing particular aspects only and is not intended to be limiting.

The term “sample” or “test sample” as used herein may refer to any material in which the presence or amount of a target analyte is unknown and can be determined in an assay. The sample may be from any source, for example, any biological (e.g. human or animal samples, including clinical samples), environmental (e.g. water, soil or air) or natural (e.g. plants) source, or from any manufactured or synthetic source (e.g. food or drinks). The sample may be comprised or is suspected of comprising one or more analytes. The sample may be a “biological sample” comprising cellular and non-cellular material, including, but not limited to, tissue samples, urine, blood, serum, other bodily fluids and/or secretions.

The term “target”, “analyte” or “target analyte” as used herein may refer to any agent, including, but not limited to, a small inorganic molecule, small organic molecule, metal ion, biomolecule, toxin, biopolymer (such as a nucleic acid, carbohydrate, lipid, peptide, protein), cell, tissue, microorganism and virus, for which one would like to sense or detect. The analyte may be either isolated from a natural source or is synthetic. The analyte may be a single compound or a class of compounds, such as a class of compounds that share structural or functional features. The term analyte also includes combinations (e.g. mixtures) of compounds or agents such as, but not limited, to combinatorial libraries and samples from an organism or a natural environment.

The term “antibody” as used herein refers to a glycoprotein, or antigen-binding fragments thereof, that has specific binding affinity for an antigen as the target analyte. Antibodies can be monoclonal and/or polyclonal antibodies. Antibodies can be chimeric or humanized.

The term “DNAzyme” as used herein may refer to a nucleic acid molecule or oligonucleotide sequence that can catalyze or initiate a reaction, optionally in response to specifically recognizing to a target analyte. DNAzymes may be single-stranded DNA, and may include RNA, modified nucleotides and/or nucleotide derivatives.

The term “organosilane” as used herein refers molecules comprising organic functional groups (i.e. hydrocarbons) which have at least one direct bond between a silicon atom and a carbon atom in the molecule. For the purpose of silanization with an organosilane, the compound also comprises at least one group bonded to the silicon atom that can be displaced for formation of a covalent bond with another entity.

In understanding the scope of the present application, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies. In addition, all ranges given herein include the end of the ranges and also any intermediate range points, whether explicitly stated or not.

As used in this application, the singular forms “a”, “an” and “the” include plural references unless the content clearly dictates otherwise.

In embodiments comprising an “additional” or “second” component, the second component as used herein is chemically different from the other components or first component. A “third” component is different from the other, first, and second components, and further enumerated or “additional” components are similarly different.

The term “and/or” as used herein means that the listed items are present, or used, individually or in combination. In effect, this term means that “at least one of” or “one or more” of the listed items is used or present.

The abbreviation, “e.g.” is derived from the Latin exempli gratia and is used herein to indicate a non-limiting example. Thus, the abbreviation “e.g.” is synonymous with the term “for example.” The word “or” is intended to include “and” unless the context clearly indicates otherwise.

It will be understood that any component defined herein as being included may be explicitly excluded by way of proviso or negative limitation, such as any specific compounds or method steps, whether implicitly or explicitly defined herein.

Although methods and materials similar or equivalent to those described herein can be used in the practice or testing of this disclosure, suitable methods and materials are described below.

### II. Compositions and Methods of the Application

The present application discloses a biofunctionalized surface, such as a biosensing interface that enables detection of biomolecular target analytes, for example, sub picogram detection of IL-6 in human plasma and/or in coagulating human whole blood. In some embodiments, the biofunctionalized surface and/or biosensing interface comprises a pattern, for example, micro/nano arrays of various target-specific probes (e.g. DNA, antibodies, etc.) for multiplex detection of target analytes.

Advantages of the present disclosure include: (i) significantly higher sensitivity for detection of IL-6 in complex biofluids such as human whole blood and plasma, (ii) simplicity of the design using ELISA-IFA, eliminating any need for use of nanoparticles, nanotubes, nanowires, fibers, and microfluidics—consequently, allowing for a low cost device that can be mass produced in a short run—(iii) robustness of the biosensor through the covalent immobilization of the capture antibodies onto the FS treated surfaces and (iv) potential capability for multiplex detection of cytokines via creation of microarrays of different biorecognition elements.

The present application includes:
1. A method for fabricating a biofunctionalized surface on a substrate, wherein the substrate comprises hydroxyl groups on the surface to be biofunctionalized, the method comprising:
   (a) covalently attaching organosilane groups to less than all of the hydroxyl groups on the surface of the substrate;
   (b) covalently attaching one or more biospecies to the surface of the substrate; and
   (c) applying a lubricant to the substrate,
   wherein the biospecies comprises a biorecognition element that detects a target analyte in a sample.
2. The method of embodiment 1, wherein the organosilane groups are attached to less than all of the hydroxyl groups on the surface of the substrate in (a) by contacting the substrate with an organosilanating reagent for about 5 minutes to about 30 minutes at a temperature of about 20° C. to about 90° C. to provide unmodified hydroxyl groups and modified hydroxyl groups and the biospecies is covalently attached in (b) to the unmodified hydroxyl groups.
3. The method of embodiment 1, wherein the organosilane groups are attached to less than all of the hydroxyl groups on the surface of the substrate in (a) by first treating the substrate with CO₂ plasma under conditions to convert only a portion of the hydroxyl groups to carboxyl groups and covalently attaching organosilane groups to the unconverted hydroxyl groups, and the biospecies is covalently attached in (b) to the carboxyl groups.
4. The method of any one of embodiments 1 to 3, wherein covalently attaching organosilane groups comprises chemical vapor deposition or liquid phase deposition.
5. The method of any one of claims 1 to 4, wherein covalently attaching the biospecies comprises applying a covalent crosslinking agent to the substrate before applying the biospecies to the substrate.
6. The method of any one of embodiments 1 to 4, wherein covalently attaching the biospecies comprises combining a covalent crosslinking agent with the biospecies into a mixture then applying the mixture to the substrate.
7. The method of any one of embodiments 1 to 6, wherein covalently attaching the biospecies comprises positioning the biospecies in a distinct pattern on the surface.
8. The method of any one of embodiments 1 to 7, wherein covalently attaching the biospecies comprises non-contact printing, optionally inkjet printing and/or spraying.
9. The method of any one of embodiments 1 to 7, wherein covalently attaching the biospecies comprises contact printing, optionally microcontact printing, roll-to-roll printing and/or stamping.
10. The method of any one of embodiments 1 to 9, wherein the substrate comprises a metallic, polymeric and/or glass material, optionally a nanoparticle.
11. The method of any one of embodiments 1 to 10, wherein the organaosilane is a fluorosilane.
12. The method of embodiment 11, wherein the fluorosilane comprises 1H,1H,2H,2H-perfluorooctyltriethoxysilane, trichloro(1H,1H,2H,2H-perfluorooctyl)silane, heptadecafluoro-1,1,2,2-tetrahydrodecyl trichlorosilane and/or 1H,1H,2H,2H-perfluorodecyltrimethoxysilane.
13. The method of any one of embodiments 1 to 10, wherein organosilane groups comprises n-propyltrichlorosilane, and/or methyltrichlorosilane.
14. The method of any one of embodiments 1 to 13, further comprising micro- or nano-sized structures on the surface.
15. The method of any one of embodiments 1 to 14, wherein the lubricant comprises a perfluorotrialkylamine, a perfluoroalkylether or perfluoroalkylpolyether, a perfluoroalkane, a perfluorocycloalkane, perfluoroperhydrophenanthrene (PFPP) and/or a perfluorohaloalkane.
16. The method of embodiment 5 or 6, wherein the covalent crosslinking agent comprises a silane coupling agent.
17. The method of embodiment 16, wherein the silane coupling agent comprises a mono-, di- or tri-functional silane.
18. The method of embodiment 16 or 17, wherein the silane coupling agent is selected from (3-aminopropyl)triethoxysilane (APTES), (3-aminopropyl)trimethoxysilane (APTMS), 3-mercaptopropyl trimethoxysilane (MPTMS) and/or glycidyloxypropyl)trimethoxysilane (GLYMO).
19. The method of embodiment 5 or 6, wherein the covalent crosslinking agent comprises a carbodiimide crosslinker, glutaraldehyde, glycidyl methacrylate, hexamethylenediamine (HMDA), 1,3-diaminopropane (DAP), N-lithioethylenediamine, N-lithiodiaminopropane, an epoxy group and/or succinimide ester such as n-α-maleimidobutyryl-oxysuccinimide ester.
20. The method of any one of embodiments 16 to 19, wherein the covalent crosslinking agent comprises a polymer, optionally in combination with a silane.
21. The method of embodiment 20, wherein the polymer comprises cyclophane-containing polymers, poly(allylamine hydrochloride), poly(ethyleneimine), poly(acrylic acid), functional polyethylene glycol (PEG) (e.g. NHS-PEG), amine functional polyacrylamide, poly(ethyleneimine) (PEI), poly(allylamine hydrochloride) (PAH), and, polyallylamine, amine functional parylenes, and/or hyperbranched polyglycerol.
22. The method of any one of embodiments 1 to 21, wherein the biospecies comprises a biomolecule, virus, cell and/or tissue.
23. The method of embodiment 22, wherein the biomolecule comprises a protein, peptide and/or nucleic acid, for example wherein the biomolecule is an antibody or a DNAzyme.
24. The method of any one of embodiments 1 to 23, wherein the biospecies further comprises a nanoparticle.
25. The method of any one of embodiments 1 to 24, wherein the biospecies are positioned in a distinct pattern on the surface.
26. Use of a biofunctionalized surface prepared using a method of any one of embodiments 1 to 25 as a biosensor.
27. A biosensor comprising a biofunctionalized surface prepared using a method of any one of embodiments 1 to 25.
28. The biosensor of embodiment 27, wherein the biofunctionalized surface is capable of preventing non-specific adsorption.
29. The biosensor of embodiment 27 or 28, wherein the biosensor provides and multiplex detection of different target analytes.
30. The biosensor of any one of embodiments 27 to 29, wherein the biosensor is used for clinical and agricultural diagnostics, agri-food quality control, environmental monitoring, health screening, health monitoring, and/or pharmaceutical development.
31. A device comprising the biofunctionalized surface prepared using a method of any one of embodiments 1 to 25.
32. A device comprising the biosensor of any one of embodiments 27 to 30.
33. A biofunctionalized surface prepared using a method of any one of embodiments 1 to 25.

Accordingly, provided herein is a biofunctionalized surface comprising a substrate functionalized with a silane and a covalently-bound biospecies, wherein the biospecies comprises a biorecognition element capable of detecting a target analyte in a sample.

In some embodiments, the substrate comprises a metallic, polymeric and/or glass material, optionally a nanoparticle. In some embodiments, the substrate comprises a nanoparticle or an entity that possesses any other scale dimension topography or geometry.

In some embodiments, the silane comprises a fluorosilane. In some embodiments, hydroxylation is followed by fluorosilanization of the substrate. In some embodiments, the fluorosilane is, but not limited to, 1H,1H,2H,2H-perfluorooctyltriethoxysilane, 2-(perfluorodecyl)ethyl acrylate, 1H,1H,2H,2H-perfluorodecanethiol, trichloro(1H,1H,2H,2H-perfluorooctyl)silane, and/or 1H,1H,2H,2H-perfluorodecyltrimethoxysilane.

In some embodiments, the biofunctionalized surface further comprises a lubricant. In some embodiments, a lubricant (e.g. a fluorinated lubricant) is subsequently infused to the substrate to create omniphobic properties thereby preventing non-specific adsorption of biospecies. In some embodiments, the fluorinated lubricant is, but not limited to, perfluorotrialkylamine (e.g. a C3-perfluorotrialkylamine such as perfluorotripentylamine), a perfluoroalkylether or perfluoroalkylpolyether (e.g. a polymer of polyhexafluoropropylene oxide of the formula F—(CF(CF₃)—CF₂—O)ₘ—CF₂CF₃, wherein m is an integer of from 10 to 60), a perfluoroalkane (e.g. a C₅₋₁₂perfluoroalkane such as perfluorohexane or perfluorooctane), a perfluorocycloalkane (e.g. perfluorodecalin or perfluororperhydrophenanthrene) or a perfluorohaloalkane, wherein halo is other than fluoro (e.g. a C₅₋₁₂ perfluorobromoalkane such as bromoperfluorooctane).

In some embodiments, non-fluorinated silane, such as n-propyltrichlorosilane, is used to chemically modify the surface for infusing lubricant. In some embodiments, chemical modification and/or lubricant infusion provides omniphobicity and prevents non-specific adsorption.

In some embodiments, the chemical modification of the substrate comprises adding moieties having affinity for, or being compatible with, the lubricant, such that the lubricant is retained on the surface. For example, halo-containing moieties such as fluoro, chloro, bromo, iodo groups on each of the modification moiety and the lubricant may be contemplated. Selection of compatible modifications and lubricant would be well within the purview of a skilled person in the art.

In some embodiments, the biofunctionalized surface further comprises micro- or nano-sized structures on the surface. In some embodiments, inducing micro/nano structures onto the surface provides omniphobic properties.

In some embodiments, functionalization of the biospecies, such as biomolecules, is achieved by printing the developed bioink solution onto the omniphobic surface prior to adding the lubricant in the case that the lubricant infusion is required to obtain omniphobic properties. In some embodiments, to further promote the stabilization of, optionally patterned, biospecies through microcontact printing, covalent printing of the capture antibodies may be performed via the introduction of functional bioinks. In some embodiments, the biospecies/bioink is functionalized with a covalent crosslinking agent. In some embodiments, the covalent crosslinking agent comprises a silane coupling agent. In some embodiments, the silane coupling agent comprises a mono-, di- or tri-functional silane. In some embodiments, prior to printing onto the surface, the biospecies are functionalized with a silane coupling agent, such as, but not limited to, (3-aminopropyl)triethoxysilane (APTES), (3-aminopropyl)trimethoxysilane (APTMS), and/or 3-mercaptopropyl trimethoxysilane (MPTMS), glycidyloxypropyl)trimethoxysilane (GLYMO). Functionalization of the biospecies is conducted by covalent attachment of the tail groups of the silane coupling agents to any functional group of the biospecies (e.g. amine groups, carboxylic groups, hydrazines, hydrazides, thiol group, etc.) using a crosslinking agent (e.g. carbodiimide chemistry, glutaraldehyde, succinimide esters, etc.) if needed. In some embodiments, the crosslinking agent comprises a carbodiimide crosslinker, glutaraldehyde and/or succinimide ester. In some embodiments, 1-Ethyl-3-(3 dimethylaminopropyl) carbodiimide (EDC), N′, N′-dicyclohexyl carbodiimide (DCC) or N,N′-diisopropyl carbodiimide (DIC) and N-hydroxysuccinimide (NHS) and sulfo-NHS are used for activation of carboxylic groups.

In some embodiments, the developed bioink comprises a polymer, optionally in combination with a silane. In some embodiments, the developed bioink is made by functionalization of the biospecies with polymers such as cyclophane-containing polymers, poly(allylamine hydrochloride), hexamethylenediamine, 1,3-diaminopropane, poly(ethyleneimine), poly(acrylic acid), functional polyethylene glycol (PEG) (e.g. NHS-PEG), amine functional polyacrylamide, and/or hyperbranched polyglycerol.

The biofunctional biospecies then covalently bind to the free hydroxyl groups on a functionalized omniphobic surface through the hydroxyl groups of the silane coupling agent at its head groups and forming oxane/siloxane bonds. The free hydroxyl groups of the surface are resulted by selective or incomplete fluorosilanization of the surface.

In some embodiments, a secondary hydroxylation step (e.g. secondary O₂/CO₂ plasma treatment, UVO treatment, piranha etching, etc.) is performed after creating the omniphobic surface to increase the amount of hydroxyl groups for better attachment of the developed bioink. In some embodiments, a secondary plasma treatment is performed to create amine functional groups onto the omniphobic surface using allylamine plasma, ammonia plasma, and/or nitrogen plasma. In some embodiments, the induced amine functional groups are then bound to the developed bioink via a crosslinking agent (e.g. use of carbodiimide chemistry).

In some embodiments, the biospecies are positioned in a distinct pattern on the surface. In some embodiments, covalent patterning of the biospecies on the omniphobic surface is achieved by non-contact printing methods (e.g. inkjet printing techniques), contact printing methods (e.g. microcontact printing techniques using PDMS stamps or other types of stamps), and other methods such as microfluidic gradient generators.

In some embodiments, these biofunctionalized surfaces promote covalent binding to the FS surface with various biospecies through their amine moieties using the disclosed bioink preparation technique, which facilitates the transfer of biospecies (e.g. biomolecules, such as antibodies) from a PDMS stamp to the FS-treated PMMA substrate, resulting in a higher yield of biospecies immobilized onto the surface.

In some embodiments, the biospecies comprises a biomolecule, virus cell, and/or tissue. In some embodiments, the biomolecule comprises a protein, peptide and/or nucleic acid. In some embodiments, the virus comprises bacteriophage. In some embodiments, the cell comprises a prokaryotic and/or eukaryotic cell. In some embodiments, the tissue comprises decellularized tissue.

In some embodiments, the biospecies further comprises a nanoparticle. IN some embodiments, the nanoparticle is, but not limited to, a magnetic nanoparticle, TiO₂, MnO₂, silver nanoparticle, polymeric-based nanoparticle, a hydrogel, natural nanoparticle and/or lipid-based nanoparticle.

The disclosed biosensing interface benefits from the repellency and omniphobicity of the LIS, which blocks non-specific attachment of interfering matrix components to the surface, thereby enhancing the sensitivity and specificity of the biosensor.

In some embodiments, microcontact printing of a bioink and lubricant-infusion of a fluorosilanized surface are combined to develop a biosensing interface for covalently attaching IL-6 capture antibody onto a poly(methyl methacrylate) (PMMA) substrate. In some embodiments, the bioink, comprises an epoxysilane anti-IL-6 complex, enabling covalent microcontact printing of the capture antibody onto the FS PMMA surface. In some embodiments, this provides a robust and stable immobilized capture antibody, that enhances the selectivity and reproducibility of an IL-6 IFA while achieving low LOD.

In some embodiments, the biosensing interface comprises an IFA the sensing platform. In some embodiments, the biosensing interface comprises a bead or nanoparticle-based enzyme-linked immunosorbent assay IFA (ELISA-IFA). In some embodiments, applying the LIS anti-fouling coating to the bioink printed PMMA surfaces produced a robust, simple and cost-effective IFA that allowed detection of IL-6 in human whole plasma with an LOD as low as 0.5 pg mL⁻¹ and enabled detection in citrated human whole blood during its coagulation induced by calcium chloride. In some embodiments, the presence of epoxy as a cross-linking agent in the bioink facilitates a higher yield of antibody immobilized onto the surface and provides robustness due to the covalent bond between the antibody and the surface.

In some embodiments, the biosensing interface comprises DNAzyme. In some embodiments, the DNAzyme is “RNA-cleaving” and catalyzes the cleavage of a particular nucleic acid molecule, for example a nucleic acid sequence comprising one or more ribonucleotides, at a defined cleavage site. In some embodiments, the molecule is a target nucleic acid in a sample. In some embodiments, the DNAzyme cleaves a single ribonucleotide linkage. In some embodiments, the single ribonucleotide linkage is in a nucleic acid sequence wherein the remaining nucleotides are ribonucleotides. In some embodiments, the single ribonucleotide linkage is in a nucleic acid sequence wherein the remaining nucleotides are deoxyribonucleotides. In some embodiments, the DNAzyme cleaves a nucleic acid sequence at a single ribonucleotide linkage thereby producing a nucleic acid cleavage fragment.

Accordingly, also provided herein is a biosensor comprising the biofunctionalized surface disclosed herein. In some embodiments, biofunctionalized surface of the biosensor is capable of effectively preventing non-specific adsorption. In some embodiments, the biosensor provides and multiplex detection of different target analytes. In some embodiments, the biosensor is used for clinical and agricultural diagnostics, agri-food quality control, environmental monitoring, health screening, health monitoring, and/or pharmaceutical development.

Accordingly, also provided herein is a device comprising the biofunctionalized surface or biosensor disclosed herein.

Accordingly, also provided herein is use of the biofunctionalized surface, biosensor or device disclosed herein.

Accordingly, also provided herein is a method for fabricating a the biofunctionalized surface disclosed herein, the method comprising hydroxylating the substrate, silanating the substrate, covalently attaching a biospecies onto the substrate, and optionally applying a lubricant onto the substrate, wherein the biospecies comprises a biorecognition element capable of detecting a target analyte in a sample.

In some embodiments, the method is used to create a bio-functional lubricant-infused surface capable of being covalently micro/nano patterned with a desired biospecies. In some embodiments, the method allows for covalent micro/nano patterning of different biological entities (i.e. biospecies) with amine or silane moieties onto a fluorosilanized or plain substrate so as to produce functional patterned lubricant-infused surfaces. In some embodiments, the method comprises covalent microcontact printing of bio molecules onto a hydrophobic (e.g. FS coated) surface. While this technique provides an omniphobic surface which effectively blocks any non-specific attachment to the surface, as well as self-cleaning and repellency properties, it remains functional for targeted binding to biospecies as a result of micro/nano patterning of various biospecies, nanoparticles or other entities with functional moieties on the surface.

In some embodiments, hydroxylating the substrate (e.g. metallic/polymeric/glass substrates) is performed first using different methods such as, but not limited to, plasma treatment, piranha etching, Ultraviolet/Ozone treatment, corona discharge. In some embodiments, plasma treatment comprises using gaseous air, O₂, CO₂ or a combination thereof. In some embodiments, silanating the substrate comprises chemical vapor deposition or liquid phase deposition. In some embodiments, silanating the substrate comprises deposition of a fluorosilane. In some embodiments, silanating the substrate comprises covalently attaching organosilane groups to less than all of the hydroxyl groups on the surface of the substrate. In some embodiments, covalently attaching organosilane groups to less than all of the hydroxyl groups on the surface of the substrate comprises attaching to about 40% to about 70% of the hydroxyl groups. In some embodiments, organosilane groups are attached to about 50% to about 70%, or about 60% to about 70% of the hydroxyl groups. In some embodiments, conditions for covalently attaching organosilane groups to less than all of the hydroxyl groups on the surface comprises a reaction time from about 5 minutes to about 30 minutes and a temperature from about 20° C. to about 90° C. In some embodiments, the reaction time is from about 10 minutes to about 30 minutes, or from about 15 minutes to about 30 minutes. In some embodiments, the temperature is from about 30° C. to about 90° C., or from about 40° C. to about 90° C., or from about 60° C. to about 90° C.

In some embodiments, the method further comprises hydroxylating the surface after silanating the substrate. In some embodiments, the method further comprises plasma treatment after silanating the substrate. In some embodiments, the plasma treatment comprises gaseous air, O₂, CO₂, allylamine plasma, ammonia plasma, and/or nitrogen plasma. In some embodiments, covalently attaching a biospecies comprises applying a covalent crosslinker to the substrate before applying the biospecies to the substrate. In some embodiments, covalently attaching a biospecies comprises combining a covalent crosslinker with the biospecies into a mixture then applying the mixture to the substrate. In some embodiments, covalently attaching a biospecies comprises positioning the biospecies in a distinct pattern on the surface. In some embodiments, covalently attaching a biospecies comprises non-contact printing, optionally inkjet printing and/or spraying. In some embodiments, covalently attaching a biospecies comprises contact printing, optionally microcontact printing, roll-to-roll printing and/or stamping. In some embodiments, the covalently attaching a biospecies comprises using microfluidic gradient generators. In some embodiments, covalently attaching a biospecies is performed after addition of a lubricant.

In some embodiments, the biospecies are bound to the surface via carbodiimide chemistry. In some embodiments, the procedure starts with CO₂ plasma treatment of substrates which can selectively or partially create carboxylic groups on the surfaces. The surface is then silanated to functionalize the free hydroxyl groups and the biospecies bound to a linker are covalently attached to the carboxyl groups. In some embodiments, using a sulfuric acid/hydrogen peroxide etchant creates carboxylic groups on the surface. The hydroxyl groups are then brought into contact with fluorosilane molecules during a chemical vapor deposition step to form fluorosilanized surfaces while the carboxylic groups are remained for further activation. Using an inkjet printer, carboxylic groups via printing EDC-NHS can be activate locally. The distinct activated areas can subsequently bind to the amine groups of the desired biospecies, nanoparticles or other substrates with amine moieties to form micro/nano patterned bio-functional surfaces. In addition, the rate of the fluorosilanization can be controlled so that free hydroxyl groups remain available following fluorosilanization. These free remaining hydroxyl groups can be used to attach silanes or silanized-entities such as silanized biospecies and particles. Finally, infusing a fluorocarbon lubricant into the surface brings about a monolayer of lubricant blocked onto the fluorosilanized surface with superior omniphobicity and repellency properties. This eliminates the need for any other blocking agent since the lubricant-infused surfaces can more effectively prevent any non-specific binding. The fabrication method is simple and scalable for mass production. Moreover, the covalently micro/nano patterning method provides a robust biofunctional surface to be used in harsh environment.

In some embodiments, if surface blocking is not required, the explained process can be performed without fluorosilanization of the surface following CO₂ plasma treatment. In this case, EDC-NHS can be microcontact printed alone onto the surfaces, activating the carboxylic groups which can subsequently react with the amine groups of the desired entity.

In some embodiments, it is possible to first mix EDC-NHS with the entity of interest and then directly micro/nano print the entity onto the treated surface. Although this may lead to self-binding of the entity and partial waste of it, the method could eliminate the step required for printing EDC-NHS separately thereby accelerating the fabrication process.

In some embodiments, the method may be modified to enable other entities with different functional groups to bind to the treated surfaces. For example, using different gases in plasma treatment step such as O₂, air etc. or a combination of gasses make it possible to induce different functional groups onto the surfaces which could later be utilized to anchor entities with certain moieties such as epoxy or silane.

In some embodiments, the method allows for covalent micro patterning of the FS surface with a desired capture antibody wherein the stability of the immobilized biomolecules is significantly increased.

The disclosed method is robust, simple, and scalable for mass production and can be applied to different substrates and for the detection of other target analytes. In some embodiments, the method allows for mass production of the developed biosensing interfaces as well as enabling multiplex detection of target analytes, such as disease biomarkers.

In some embodiments, the method provides a substrate covalently coated with a monolayer of fluorosilane wherein the repellency behavior of the surface is more durable in comparison to common blocking agents that are physically or electrostatically attached to the surface.

### EXAMPLES

The following non-limiting examples are illustrative of the present application:

### Example 1. Fluorosilanization and Micro/Nano Printing of Surfaces

Glass microscope slides as well as polystyrene substrates were used as substrates. Samples were first washed with ethanol and then placed in a plasma machine. CO₂ plasma treatment was performed for 5 min. The samples were moved to a vacuum desiccator for the subsequent chemical vapor deposition (CVD) step. CVD treatment was carried out with 200 μl of trichloro (1H,1H,2H,2H-perfluorooctyl) silane for 1 hour. Next, the samples were heat treated at ˜100° C. for 1 hour.

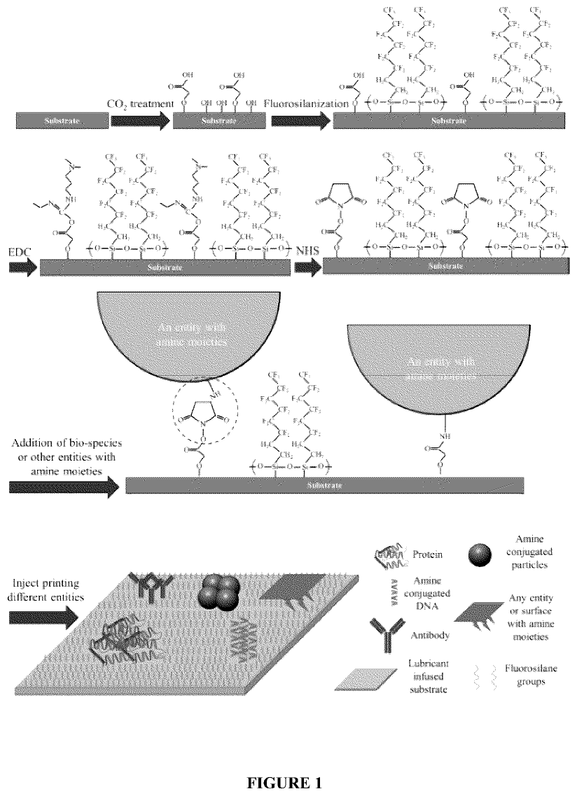

FIG. 1 shows bio-interface fabrication by micro/nano patterning different entities such as DNA, antibodies, proteins, micro/nano particles, and surfaces with amine moieties onto a lubricant-infused surface. Moreover, different entities can be covalently immobilized on one single substrate by using an inkjet printer.

Two optional approaches may be used for (bio)functionalization. Approach one: A mixture of 1-Ethyl-3-(3 dimethylaminopropyl) carbodiimide (EDC) and N-hydroxysuccinimide (NHS) was employed for activating the carboxylic groups remained after the fluorosilanization process. EDC-NHS with the molar ratio of −1:1 diluted in MES buffer was inkjet printed as micro dots onto the surfaces. After that, the samples were incubated in a humidity chamber for 30 min. Then, the samples were washed with water and added fluorescein isothiocyanate (FITC) conjugated bovine serum albumin (BSA) diluted in PBS to the entire surface and incubated the samples for at least 1 hour.

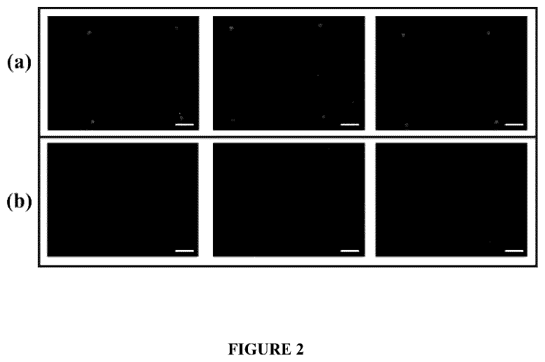

Fluorescent images of the surfaces micro patterned with EDC-NHS and then incubated with FITC-labeled BSA (BSA-FITC) In comparison to the CO₂ treated surfaces, the bright spots could not be observed in the control sample (O₂ plasma) confirming the importance of CO₂ plasma (FIG. 2). The lack of carboxylic groups on the O₂ plasma treated samples did not provide enough activated areas to bind to the BSA-FITC.

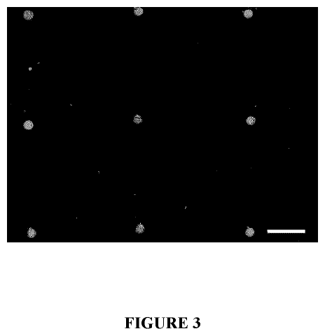

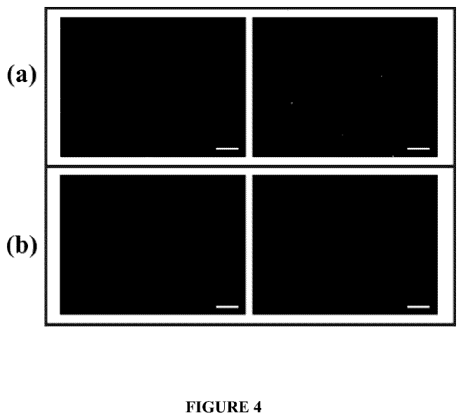

In FIG. 3, surfaces treated with CO₂ plasma before the salinization process indicate a possible distance reduction between patterned areas which is highly beneficial for high throughput applications. Patterns were not formed well in FIG. 4 demonstrating the importance of a mixture of EDC and NHS for coating.

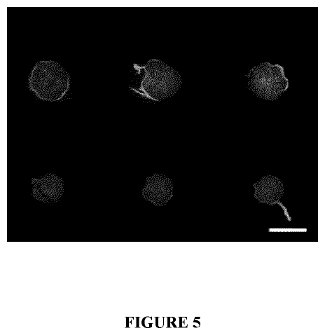

Approach two: EDC-NHS with the molar ratio of ˜1:1 was first mixed with BSA-FITC diluted in PBS before inkjet printing. The solution was added to the surfaces via inkjet printer and the incubation was done for more than 1 hour in a humidity chamber. The results in FIG. 5 demonstrate higher fluorescence intensity compared to the first approach where EDC-NHS were printed separately.

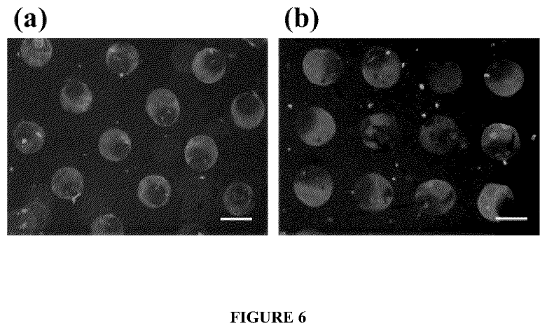

Using approach two, the amine conjugated fluorescently labeled DNA was immobilized on two different substrates of glass and polystyrene. DNA sample was diluted in MES buffer containing EDC-NHS with the molar ration of ˜1:1. The solution immediately inkjet printed onto the substrates and the incubation was done overnight. This demonstrates that the procedure can be applied on various substrates using different biospecies, such as biomolecules (e.g. proteins, nucleic acids, etc.), with amine moieties (FIG. 6).

Finally, for both approaches, the samples were harshly washed with TBS-Tween 20 buffer before the imaging was performed to remove non-covalent attached proteins. It should be noted that the size of the droplets in the inkjet printing step could easily be adjusted to obtain either micro or nano patterns of the desired biospecies (e.g. biomolecules).

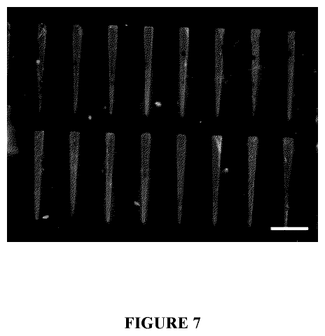

For microcontact printing EDC-NHS, clean microscope glass slides were CO₂ plasma treated for 5 min. Next, EDC-NHS with the molar ratio of ˜1:1 diluted in MES buffer was microcontact printed onto the surfaces. Then CD34 diluted in PBS was added to the entire surface and incubated for about 1 h. The surfaces were washed with TBS-Tween 20 buffer before imaging. FIG. 7 demonstrates the possibility to activate the carboxylic groups by just microcontact printing EDC-NHS.

### Example 2. Biosensor Interface Biofunctionalization and Immunoassay

Methods.

The following materials and reagents have been utilized for surface biofunctionalization and IL-6 sandwich immunoassay: trichloro(1H,1H,2H,2H-perfluorooctyl)silane (TPFS) (Sigma-Aldrich, Oakville, ON, Canada), (3-glycidyloxypropyl)trimethoxy-silane (GLYMO) (Sigma-Aldrich, Oakville, ON, Canada), perfluoroperhydrophenanthrene (PFPP) (Sigma-Aldrich, Oakville, ON, Canada), polydimethylsiloxane (PDMS) (Dow SYLGARD™ 184 Silicone Encapsulant, Ellsworth Adhesives, Stoney Creek, ON, Canada), recombinant human (E. Coli derived) IL-6 (R&D Systems, Minnesota, US), IL-6 monoclonal antibody (MQ2-13A5, capture antibody) (ThermoFisher Scientific, ON, Canada), biotinylated IL-6 monoclonal antibody (MQ2-39C3, detector antibody) (ThermoFisher Scientific, ON, Canada), BV480 streptavidin (BD Horizon™, Mississauga, ON, Canada), and Poly(methyl methacrylate) plates (PMMA) (Beauty Glass, Hsin Hwa Chemical Co, Ltd, Taiwan). Human blood plasma, and citrated whole human blood were used as received without any modification and collected from healthy donors who provided a signed consent for collecting their blood.

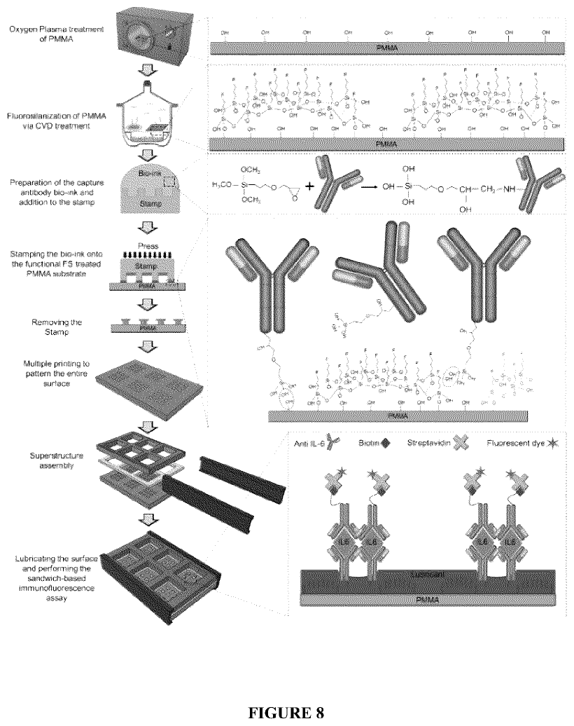

Surface fluorosilanization. Before functionalization of PMMA, the substrates were cut to the size of a glass slide (75 mm×25 mm), and carefully washed using water and ethanol to remove any traces of impurities. The PMMA surface was initially oxygen plasma treated for 15 min (Harrick Plasma) to hydroxylate the surface. Afterwards, the substrate was immediately transferred to a vacuum desiccator in order to perform fluorosilanization through CVD method using trichloro(1H,1H,2H,2H-perfluorooctyl)silane for 30 mins at −0.08 MPa pressure. Subsequently, the fluorinated surface was placed on a hot plate at 90° C. for 30 mins to create FS SAM (FIG. 8).

Preparation and microcontact printing of the bioink. (3-glycidyloxypropyl)trimethoxy-silane was diluted in PBS to achieve the concentration of 22.4 μM. Then, IL-6 monoclonal capture antibody was mixed with the epoxy solution at the concentration of 150 μg/mL. The prepared bioink was patterned on the fluorosilanized PMMA surface through microcontact printing. In order to make PDMS stamps, a silicon wafer mold with the desired patterns was fabricated using photolithography technique. PDMS was cast into a mold to produce stamps with an array of square protrusions (50×50 μm²). The stamps were sonicated in ethanol and dried before use. The required amount of the capture antibody bioink (˜5 μl) was added to each stamp to cover the entire features of the stamps and incubated for 10 mins. Thereafter, the stamps were washed with PBS and then water and dried for a 2-4 seconds using a strong blast of compressed air. The stamps were immediately pressed onto the FS treated PMMA surface by placing a small amount of weight on top. After 2 mins, the stamps were removed, and the surfaces were incubated in a humidified atmosphere at 4° C. for around 1 hr and a half

Lubricant-infusion of the fluorosilanized surface. Following the covalent antibody printing onto the fluorosilanized surface and assembly of the superstructure (FIG. 8), PFPP lubricant was added to the wells and incubated for about 1-2 min. Then, the surface was flipped to remove the excess amount of the lubricant and washed with Tris buffered saline (TBS) and TBS Tween 20 (TBST) leaving a thin layer of lubricant trapped within the fluorine groups of the surface.

Surface characterization. High-resolution X-ray photoelectron spectroscopy (XPS) (PHI Quantera II, Biointerfaces Institute, McMaster University) was implemented to reveal the chemical bonds which appeared on the surface before and after the plasma treatment and after the fluorosilanization step. XPS was performed 1 week after the surface treatment to ensure the chemicals and functional groups of the modified surfaces remain stable. C1s, O1s, and F1 s peaks were recorded in the high resolution XPS analysis and the raw data was deconvoluted incorporating CasaXPS application.

The hydrophobicity and surface tension of the PMMA substrates were quantified using contact angle and sliding angle techniques. The contact angles of plain, oxygen plasma treated, and fluorosilanized PMMA surfaces were measured via Future Digital Scientific OCA20 goniometer (Garden City, NY). In these experiments, 2 μl of deionized water was dropped onto the multiple spots of the surfaces and the contact angles were automatically calculated from the captured images of the droplets. A digital angle level (ROK, Exeter, UK) was adopted to measure the sliding angles of the plain and fluorosilanized PMMA samples. Before adding 2 μl of deionized water to the surfaces and obtaining the sliding angles, both plain and FS treated surfaces were lubricated with the PFPP lubricant and tilted to remove the excess amount of the lubricant. The droplets were placed onto the substrates at various angles, and the angle at which the droplet starts sliding off the surface was assigned as the sliding angle.

IL-6 immunofluorescence assay (IFA). Recombinant IL-6 solution was serially diluted in either sample diluent composed of 1% BSA in phosphate-buffered saline (PBS) or human blood plasma to produce the different concentrations of 2500 pg mL⁻¹, 312.5 pg mL⁻¹, 156 pg mL⁻¹, 40 pg mL⁻¹, 20 pg mL⁻¹, 5 pg mL⁻¹, 2 pg mL⁻¹, 1 pg mL⁻¹, 0.8 pg mL⁻¹, and 0.5 pg mL⁻¹. 150 μl of the IL-6 solution was added to each well and incubated for an hour. Next, the wells were washed again using TBS and TBST, and biotinylated IL-6 monoclonal antibody (1:500 v:v diluted in the sample diluent buffer) was added to the wells and incubated for an hour. Finally, the wells were washed using both wash buffers, and the BV480 streptavidin dye (1:250 v:v diluted in the reporter buffer) was added to each well and incubated for 30 mins in complete darkness. The well plate was washed with the wash buffers before the imaging.

Fluorescence microscopy. Fluorescent imaging was conducted via a Zeiss inverted fluorescent microscope (AX10) using Fluorescein (FITC) filter set. The images were acquired via ZEN software under 10× magnification. In Fiji ImageJ software, the acquired 16-bit TIF images were first divided into individual colors (Image Color Split Channels). Next the Image Adjust Threshold function was used to define a printed square's positive signal. Square command was used to enclose a square around the PDMS stamped region. The pattern of squares for each well is replicated between images with the Take command of the ROI Manager. The median MFI signal intensities were then obtained by using the Measure command. Four smaller squares were then overlaid on the blank regions between the printed squares to obtain the background signal which was then subtracted from the MFI of the patterned areas for each well. The resulting raw data is processed by averaging the 9 positive and 4 negative spots for each image. Duplicates of each sample, control or standard yielding 18 replicate squares for each measurement were evaluated on the IL-6 IFA PMMA slides,

In Chan-Vese segmentation image processing in Python, pixels within a border demark the spot region while pixels outside the border belong to local background. Spot signal is calculated as the MFI of spot region minus the median pixel value of local background (i.e., background subtraction). For each level, a ‘raw’ mean and standard deviation are calculated from the signals of 18 replicates. In addition, Tukey Biweight algorithm has been applied on the raw data to obtain the robust mean and standard deviation (i.e., weighted mean & STD) in order to minimize the influence of outliers.

The two-tailed student T-test assuming unequal variance was used for analysis of significant differences between IL-6 levels of the dose response curve. The fitting trendlines in the dose respond graph was done by a logarithmic relationship of Equation 1 (MFI=a×ln(x)+b), where x is the IL-6 concentration in pg mL⁻¹, and a and b are the fitting parameters. The number of tests for each concentration was 3.

Blood clot formation and scanning electron microscopy (SEM) imaging. By initiating blood clot formation onto PMMA substrates, the blood cells and proteins interactions were assessed with modified lubricant-infused surfaces compared to untreated PMMA surfaces. In order to initiate the clot, 100 μl of the citrated human blood was recalcified by 100 μl of 25 mM CaCl₂) (diluted in HEPES) and added to each of the wells with the treated and untreated PMMA bottom surfaces. After 1 hr incubation, the clot was gently removed, and the wells were washed several times with TBS and TBST wash buffers. Further, 2% glutaraldehyde (diluted in PBS) was added to the wells and incubated overnight to fix the clot. After proper washing, the surfaces were incubated for 1 hr with 1% osmium tetroxide in 0.1 m sodium cacodylate buffer to proceed with the post-fixation. The surfaces were dehydrated through a graded series of ethanol, and then critical point drying was performed on the samples by Leica EM CPD300 dryer (Leica Mikrosysteme GmbH, Wien, Austria) using liquid CO₂ flush. The samples were then sputtered with gold (Polaron Model E5100 sputter coater, Watford, Hertfordshire) and imaged via SEM (JSM-7000 F).

Results

Antibody embedded lubricant-infused surfaces were created on PMMA, a low-priced polymer with many advantages such as optical transparency, durable chemical and mechanical properties, and recyclability.[66,67] PMMA surfaces were first fluorosilanized via chemical vapor deposition (CVD) of trichloro(1H,1H,2H,2H-perfluorooctyl)silane followed by heat treatment at 90° C. to promote the hydrolysis and condensation reactions forming a semi-crystalline single molecular self-assembled monolayer (SAM) of fluorosilane (FS) on the surface (FIG. 1). Prior to CVD treatment, PMMA surfaces were oxygen plasma treated to induce hydroxyl groups required for the FS reaction.

The developed bioink was prepared by serial dilution of (3-Glycidyloxypropyl)trimethoxy-silane (GLYMO) in PBS and mixing the silane solution with the IL-6 capture antibody as described above. Following the FS treatment of the PMMA, the GLYMO-conjugated bioink was patterned onto the surface via microcontact printing using polydimethylsiloxane (PDMS) stamps (50×50 μm² arrays of protruded squares). The produced positional microarrays in a microtiter plate format provides improved repeatability of the assay by providing numerous sample replicates inside each well. This plays an important role in the assay performance, as well as its adaptability to high throughput applications. Moreover, since the capture antibody along with all the surface functional groups covalently bind to the surface, the developed PMMA surfaces are very robust. The biofunctional PMMA microarrays were fitted into a superstructure which provides wells for IFA (FIG. 8). The wells were subsequently lubricated via perfluoroperhydrophenanthrene (PFPP), a fluorinated lubricant that infuses into the FS SAM and is locked through Van Der Waals forces on the fluorine groups of FS SAM creating the LIS. This provides the surface with anti-fouling properties against biofluids and proteins thereby diminishing the non-specific absorption and enhancing the sensitivity of the biosensor. The developed biosensing surfaces were then used for IFA assays for IL-6 detection.

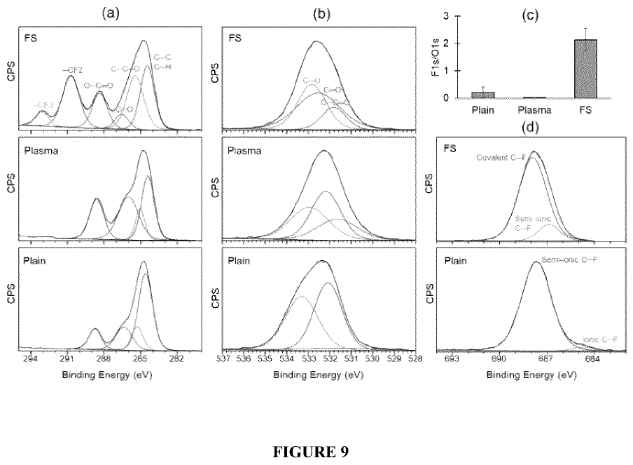

A high resolution XPS analysis was conducted on plain, plasma treated, and FS treated surfaces in order to examine the chemical bonds created after fluorosilanization of PMMA. FIG. 9 demonstrates deconvoluted spectra of C1s, O1s, and F1s for each sample. The typical deconvolution of PMMA C1s spectra is illustrated in FIG. 9 (a), plain sample, consisting of a hydrocarbon peak C—C/C—H at 284.57 (±0.1) eV, a beta carbon peak C—C═O at 285.42 (±0.16) eV, C—O bond in methoxy groups at 286.47 (±0.14) eV, and a carboxyl carbon O—C═O peak at 288.58 (±0.2) eV.[68,69] The peak area (%) of C1s components are shown in Table 1. As seen, oxygen plasma treatment raised the carboxyl carbon peak area from 12.71% to 18.91%, which could be correlated with oxidation of PMMA and formation of carboxyl groups through plasma induced oxygen radicals. Although fluorosilanization of PMMA surfaces reduced the percentage of carboxyl groups to 14.19% due to the reaction of silanol groups of the fluorosilane coupling agent, there was an increase in carboxylic groups compared to the control. Therefore, the optimized time of FS CVD treatment did not lead to the consumption of all functional hydroxyl groups on the surface. The remaining carboxylic groups provide functionality for further immobilization of anti-IL6 antibody bioink where the antibody is conjugated with the epoxy-based silane coupling agent of (3-Glycidyloxypropyl)trimethoxy-silane (GLYMO). In doing so, the trifunctional GLYMO can bind to the remaining OH groups of the FS treated surface through their silanol head groups creating C—O—Si bonds. The epoxy moiety at the tail group of the silane had been coupled with the primary and secondary amines of the antibody during the bio-ink formation. Limiting the CVD of FS to 30 minutes enables the presence of free OH groups on the PMMA surface. In fact, higher CVD durations were shown to lead to an imperfect microcontact print of the capture antibody onto the treated PMMA surfaces. In addition, considering the reasonable values of bond lengths and angles that silanol bonds can only take, binding the silane agent to the substrate with all head groups is sometimes impossible.[70,71] Thus, GLYMO is also able to bind to the silanol groups of FS layer in order to covalently couple the anti-IL6 antibody with the FS treated surface (FIG. 8). Fluorosilanization of PMMA resulted in the appearance of two new peaks in the C1s spectrum at 290.69 eV and 293.05 eV which are associated with —CF₂ and —CF₃, respectively. [72,73] These peaks confirm the formation of FS layer with the linear formula of CF₃(CF₂)₅CH₂CH₂SiCl₃ as the precursor.

The O1s spectrum of plain PMMA surface in FIG. 9 (b) can be deconvoluted into primarily two peaks of C—O at 533.3 eV and C═O at 532 eV. [74-76] Plasma treatment of the surface advanced a peak at −531.6 eV attributed to O—C═O bond [77] which remained in the O1s spectrum after fluorosilanization of the surface, indicating the presence of functional OH groups on the FS treated PMMA surfaces for covalent immobilization of the subsequent antibodies. The peak area ratio of F1s/O1s is plotted in FIG. 9 (c) which reveals a substantial increase in the amount of fluorine atoms on PMMA surfaces through the FS treatment. The F1s spectra of plain PMMA surface (FIG. 9 (d)) consisted of semi-ionic C—F bond at ˜687.5 eV, as well as ionic C—F bond at 684.5 eV. [78] These bonds were completely etched and removed via oxygen plasma treatment of the surface. Fluorosilanization of the PMMA surface resulted in the emergence of covalent C—F bond at ˜688 eV [79] which depicts the robust FS SAM created onto the PMMA substrate. Notably, the XPS test was performed a month after the surface functionalization to confirm the stability of the functional groups onto the PMMA surfaces.

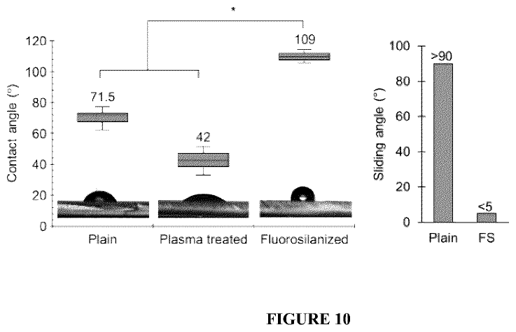

The omniphobic properties of the fluorosilanized PMMA surfaces were quantified using contact angle and sliding angle measurements (FIG. 10). The contact angle and sliding angle of plain PMMA surfaces before any treatment were estimated at 71.5° and >90°, respectively. After 15 mins of oxygen plasma treatment, the contact angle dropped to 42° due to formation of hydroxyl groups. Fluorosilanization of the PMMA surfaces for half an hour via CVD technique significantly increased the contact angle to 109°. Measuring the angular slip of the LIS estimated its interfacial tension and omniphobicity.[80,81] For this purpose, PFPP lubricant was added to the surface and the excess amount of lubricant was removed by tilting the substrate. The thin layer of the lubricant trapped onto the surface made the FS treated surfaces slippery with a sliding angle of less than 5°. For the plain PMMA surfaces, the lubricant completely came off the surface after tilting the substrate, resulting in no slippery properties. Notably, printing of the antibodies does not significantly impact the average contact angle and hydrophobicity of the PMMA surfaces as the antibody arrays occupy only a small portion of the substrate. However, due to the fact that droplets can be pinned at the printed areas, which is desirable considering the applicability of the surface for IFA, the sliding angle of the surface changes and the droplets do not slide off the entire PMMA surface. The high contact angle and low angular slip of the FS treated PMMA surfaces indicate repellent behavior of the surfaces against different proteins and biomolecules in solution, a key characteristic to prevent non-specific adhesion for biosensing.

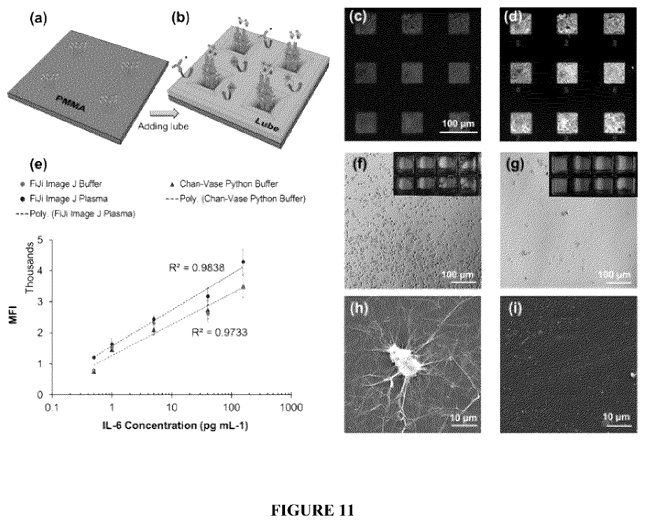

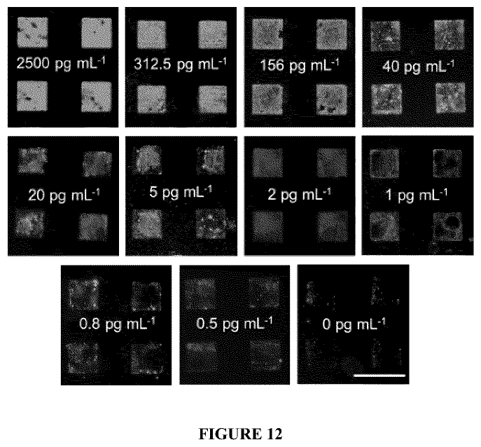

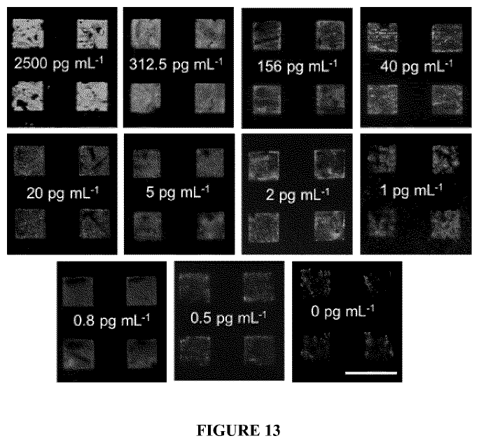

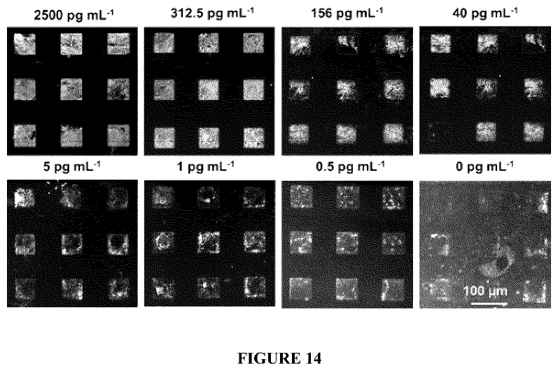

To detect IL-6 using IFA, recombinant IL-6 present in either buffer or plasma was added to the antibody printed lubricant-infused PMMA sensors at various concentrations ranging from 0 to 2500 pg mL⁻¹. Since the unprocessed human whole plasma contains several interfering biological entities such as dotting factors, hormones, albumins, and fibrinogen, detection of IL-6 in such a complex biofluids can attest to the enhanced sensitivity of the biosensor. The sandwich assay was followed by addition of a biotinylated IL-6 detector antibody and a fluorescently labeled streptavidin (FIGS. 11 (a) and (b)). Representative images of the detection fluorescent arrays at different IL-6 concentrations in buffer and plasma are shown in FIGS. 12 and 13, indicating the microarrays of IL-6 are clearly distinguishable in both plasma and buffer at the concentration of 0.5 pg mL⁻¹. Fiji ImageJ software [82] was utilized to process and quantify the mean fluorescent intensity (MFI) of each printed square. As a confirmatory method to FiJi Image J, Chan-Vese segmentation [83,84] image processing in Python was performed for quantification of results in buffer. FIGS. 11 (c) and (d), illustrates representative fluorescence images of the microarrays of IL-6 at a concentration of 312.5 pg mL⁻¹ before and after the Chan-Vese segmentation. Results of Chan-Vese segmentation processing on the other sample concentrations can be seen in FIG. 14.

Table 2 illustrates the results of the IL-6 IFA in both buffer and plasma. Two separate and individually processed imaging methods were used to confirm equivalent MFI results in buffer. The more sophisticated Chan-Vase Python approach reduces the influence of artifacts and outliers. The error values corresponding to each result is indicated as coefficient of variation (CV %) in Table 2. The LIS IL-6 IFA yielded a functional LOD of 0.5 pg mL⁻¹ which was significantly differentiated (p<5×10−6) from the 0 pg mL⁻¹ control for both raw and background-subtracted MFI values in buffer and plasma (Table 3 and 4).

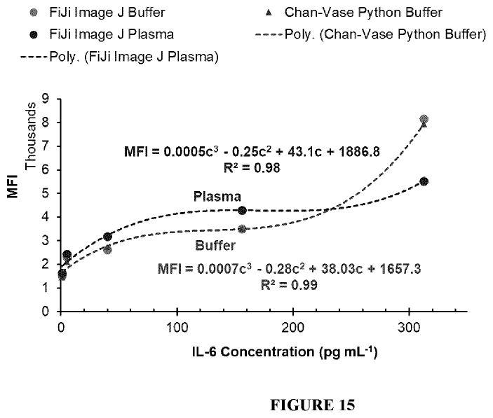

FIG. 11 (e) shows the linear dynamic range of MFI as a function of concentration. The linear range obtained form LIS IL-6 IFA after standard curve fitting was 0.5 to 156 pg mL⁻¹ with the R2>0.97. This dynamic range includes the required LOD sensitivities for oncological applications such as leukemia (1.45 pg mL⁻¹ [85]) breast cancer (3-50 pg mL⁻¹ [86]), prostate cancer (4-7 pg mL⁻¹ [87,88]), ovarian cancer (10 pg mL⁻¹ [89]), and liver cancer (12 pg mL⁻¹ [12]). Importantly, the current dynamic range is also applicable for detection of Covid-19 (SARS-Cov2) infection (5.8-64 pg mL⁻¹ [20]). Both buffer and plasma were also plotted with a quadratic (third degree polynomial) curve fit for the dynamic range of the LIS IL-6 IFA of 1 to 312.5 pg mL⁻¹ shown in FIG. 15. IL-6 detection results in plasma and buffer demonstrated almost similar MFIs, indicating that the developed biosensor is capable of preventing non-specific binding and interference in plasma enabling detection of low concentrations of IL-6 in plasma. In Table 5, the superior performance of the presented biosensor for IL-6 detection in human plasma is compared to the recent most well-known techniques along with their associated advantages and disadvantages. It is worth mentioning that the antibody-antigen interactions used as a based mechanism to capture IL-6 in this work, is intrinsically specific and it has been shown that there is not any non-specific interaction between the anti-IL-6 monoclonal antibody and other types of cytokine such as IL-2, IL-3, IL-4, IL-5, IL-8, IL-9, IL-11, TNF-α, TNF-β, IFN-α and IFN-γ.[43] Consequently, the specificity of the developed biosensor is adequately high for multiplex detection of cytokines.

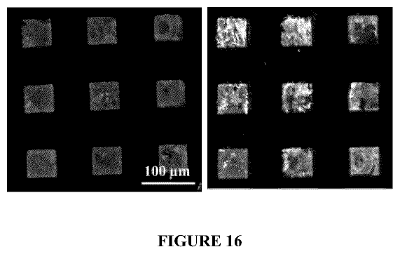

In order to evaluate the performance of the developed biosensing surface in human whole blood, the capability of the assay to recover IL-6 spiked into recalcified citrated blood was examined. The calcified blood was spiked with IL-6 at the concentration of 312.5 pg mL⁻¹ and incubated on the LIS sensing interface for an hour during blood coagulation process. Recovery of the spiked sample (n=9 replicates) on the plasma standard curve was 119.4% which is within the accuracy acceptance criteria of ±20% bias. This corresponds the upper limit of quantitation of the LIS IL-6 IFA and indicates that the assay is able to detect IL-6 in whole blood (FIG. 16). This may be the first disclosure, to the best of the inventors' knowledge, on performing biosensing in activated human whole blood while it coagulates.

In addition, a blood adhesion assay was conducted to evaluate the repellency of the surface treatment against fibrin-induced blood clot to the developed PMMA biosensing substrates. FIG. 11 (f) depicts a representative optical microscope image of blood cells attached onto the plain PMMA surface, following clot formation after an hour of incubation with recalcified citrated blood. The substrates were washed three times with tris buffered saline (TBS) and TBS Tween™ 20 (TBST) before imaging. The inset image in FIG. 11 (f) reveals the formed blood clots remaining on the plain PMMA surfaces after the washing steps. However, LIS coating significantly suppressed blood clot attachment to the surface (FIG. 11 (g)). Owing to the omniphobic characteristic of the treated PMMA surface, the clot that was formed during the blood incubation could readily be washed off the surface by the wash buffers used in the assay, while in the case of untreated PMMA surfaces, the clot remained stable onto the surface throughout the washing steps. FIGS. 11 (h) and (i) show the SEM images of the untreated PMMA surface compared to antibody embedded LIS after fixation of the remaining clot and fibrins.

Therefore, the antibody embedded lubricant-infused PMMA biosensor not only prevents non-specific adhesion of blood cells, but also enables IL-6 detection during the clot formation, facilitating accurate detection in non-anticoagulated whole blood, which may enable both ex vivo and in vivo biosensing platforms as well as biosensing in blood-contacting wearable devices. [90]

### Example 3. Biosensor Interface with DNAzymes

Methods

Materials. All DNA oligonucleotides were purchased from Integrated DNA Technologies (IDT, Coralville, IA, USA), while the TAMRA-labeled fluorogenic substrates (TS1) were purchased from Yale University. All sequences were purified via standard 10% denaturing (8 M urea) polyacrylamide gel electrophoresis (dPAGE). The sequences and functions of all synthetic oligonucleotides used herein are provided in Table 6. ATP, T4 DNA ligase, and T4 polynucleotide kinase (PNK), along with their respective buffers, were purchased from Thermo Scientific (Ottawa, ON, Canada). Plain premium microscope slides (which were used as a glass surface for DNAzyme immobilization) were obtained from VWR International (Mississauga, ON, Canada). Trichloro (1H,1H, 2H, 2H-perfluorooctyl) silane, 3-glycidyloxypropyl trimethoxysilane epoxy silane, perfluorodecalin (PFD) liquid lubricant, bovine serum albumin (BSA), and PLL-PEG were obtained from Sigma-Aldrich (Oakville, ON, Canada). Milk (2% skimmed milk, Neilson™ brand) was purchased from a local supermarket, while the water used in the experiments was purified using a Milli-Q Synthesis A10 water-purification system. All other chemicals were purchased from either Sigma-Aldrich or Bioshop Canada and were used without further purification.

Bacteria preparation. Escherichia coli K12 (E. coli K12; MG1655) was used herein. In order to measure the colony forming units (CFU/mL) of E. coli cells, a single colony that had been freshly grown on a Luria Broth (LB) agar plate was inoculated into 2 mL of LB and allowed to incubate for 14 hours at 37° C. with continuous shaking at 250 rpm. Following incubation, a 10-fold serial dilution of the bacterial culture was conducted, with 100 μL of the diluted solution being subsequently spread onto LB agar plates (done in triplicate) and incubated at 37° C. for 16 hours. Finally, the colonies were counted and averaged to obtain the number of CFU/mL. The crude intracellular mixture (CIM) of E. coli cells was prepared by centrifuging 1 mL of each dilution at 11,000 g for 5 min at 4° C. The clear supernatant was then discarded, and the cell pellet was re-suspended in 100 μL of double-deionized water (ddH₂O) and heated at 65° C. for 5 minutes. The heat-treated cell suspension was then vortexed to dissolve the cell pellet completely and stored at −20° C. The CIM of E. coli used in each experiment was based on the number of cells required for that specific experiment.

DNAzyme preparation. NH-EC1 (the amine-labeled DNAzyme sequence for E. coli K12) was enzymatically ligated to TS1 (TAMRA-labeled fluorogenic substrate) by phosphorylating 2 nmol of TS1 for 40 minutes at 37° C. in 200 μL of 1×PNK buffer A containing 2 mM ATP (final concentration) and 40 units (U) of PNK enzyme. The enzyme was inactivated by heating the reaction mixture at 90° C. for 5 minutes, and then cooling it to room temperature for 15 minutes. After cooling, an equal number of NH-EC1 and LT (ligation template) were added to the reaction mixture, which was then re-heated to 90° C. for 1 minute. The mixture was then re-cooled to room temperature for 15 minutes, at which point 40 μL of 10×DNA ligase buffer and 40 U of T4 DNA Ligase were added; the final volume was subsequently adjusted to 400 μL via the addition of ddH₂O. After incubation at room temperature for 2 hours, the DNA molecules were isolated by ethanol precipitation and the ligated DNA molecules (RFD-EC1) were purified via 10% dPAGE. The DNA molecules were then dissolved in ddH2O and the resultant DNAzyme concentration was measured using a nanoquant plate (TECAN), followed by storage at −20° C. until use. At the time of storage, the final DNAzyme concentration was 6.6 μM.

LIS surface treatment (omniphobic-lubricant-infused coating). The samples were sonicated in 70% ethanol for 10 minutes and then dried. Next, they were treated with oxygen plasma (Harrick Plasma Cleaner, PDC-002) from a 100% oxygen air liquid tank for 5 minutes in order to functionalize the surfaces. The sensors then underwent chemical vapour deposition (CVD) to create the omniphobic coating, followed by placement in a vacuum desiccator alongside a microscope slide coated with 200 μL of trichloro (1H,1H, 2H, 2H-perfluorooctyl) silane. Vacuum pressure was maintained at −0.08 MPa for 2 hours to create a self-assembled monolayer (SAM). Following CVD, the samples were cured on a hotplate for 1.5 hours at 80° C.

Epoxy activation of DNAzyme. A diluted form of 3-glycidyloxypropyl trimethoxysilane epoxy silane was used to covalently attach the DNAzyme to the surfaces. First, 5 μL of the epoxy silane was mixed with 20 μL of 1×PBS buffer (pH 7.5). 5 μL of this solution was then mixed with 1000 μL of 1×PBS. The serial dilution was completed by repeating this step for two more dilutions. The final diluted solution was used to covalently immobilize the DNAzyme onto the surfaces.

Covalent immobilization of diluted epoxy and DNAzyme onto the surfaces. Diluted epoxy silane and the DNAzyme solution were applied onto the LIS-treated surfaces using a Scienion printer. First, 400 μL of epoxy silane solution was printed onto each spot on the surface, followed by the application of 400 μL of 6.6 μM DNAzyme solution to the same spot. The epoxy and DNAzyme were printed onto the surface using a Scienion printer, and the sensors were then incubated overnight (14 hours) in a 75% humidity chamber in a dark environment. Following incubation, any unattached DNAzyme was washed off using 1×PBS buffer. The DNAzyme's covalent attachment to the surfaces was then confirmed under microscopic observation. After immobilization was confirmed, experiments were conducted on surfaces.

Determining the background fluorescent signal of milk and single-stranded DNA. Next, the green and red fluorescent background signals of milk on its own and milk mixed with either FAM- or TAMRA-labeled single-stranded DNA were determined. Briefly, 2 μL of diluted epoxy and 4 μL of amine-labeled single-stranded DNA were mixed together and immobilized onto the surface using a Scienion printer. After incubating overnight (14 hours) in a 75% humidity chamber, the surfaces were washed in a 20× saline-sodium citrate (SSC) buffer (pH 7.1). Next, 23 μL of milk and 2 μL of the complementary single-stranded DNA (either FAM- or TAMRA-labeled) were added to the surfaces. The samples were then incubated for 2 hours at room temperature in a dark environment to avoid photobleaching the fluorescent probes and to facilitate the hybridization of the two complementary strands, thus resulting in a fluorescent signal. After 2 hours, the samples were imaged using a fluorescent microscope.

Cleavage test by exposing the surfaces to contaminated milk. After printing the DNAzyme onto the surfaces and incubating overnight, the LIS treatment was completed by applying 30 μL of PFD liquid lubricant to a group of the washed samples. The samples were then submerged in 1×PBS buffer to remove any residual PFD from the surfaces. To determine the effect of PFD blocking, lubricant was not applied to one group of samples. Next, 10 μL of milk, 10 μL of 10⁶ CFU/mL of E. coli cells, and 20 μL of 2× Reaction Buffer (2×RB; 100 mM HEPES, pH 7.5, 300 mM NaCl, 30 mM MgCl₂) were added to both groups of surfaces, followed by incubation for 1 hour at room temperature (in a dark environment) to allow for cleavage to occur in the presence of bacteria. To create a control, E. coli cells were not added to one group of samples; instead, only 20 μL of milk and 20 μL of 2× reaction buffer were added to this group. After 1 hour of incubation, the samples were imaged using a fluorescent microscope.

Comparison of different blocking agents. The following blocking agents were used in the experiment: Bovine serum albumin (BSA), PLL-PEG, and PFD liquid lubricant. A 1% g/mL solution of BSA was prepared by dissolving 10 mg of BSA in 1 mL of 1×PBS buffer. Similarly, a 0.01% g/mL solution of PLL-PEG was prepared by dissolving 0.1 mg of PLL PEG in 1 mL of 1×PBS. 130 μL of BSA or PLL-PEG was added to their respective samples, following by incubation at room temperature (in a dark environment) for 30 minutes to allow for surface blocking to occur. Following incubation, the samples were submerged in 1×PBS buffer to remove any residual blocking agents. 30 μL of PFD was then applied to the lubricant sample group, following by submersion in 1×PBS buffer. To create a control, one group of samples was not subjected to any blocking agents. Next, 10 μL of milk, 10 μL of 10⁶ CFU/mL of E. coli cells, and 20 μL of 2× Reaction Buffer were applied to all the surfaces. The samples were then incubated for 1 hour at room temperature (in a dark environment) to allow for cleavage to occur in the presence of bacteria. A second control group was created by excluding E. coli cells, and only adding 20 μL of milk and 20 μL of 2× Reaction Buffer. After 1 hour of incubation, the samples were imaged using a fluorescent microscope.

Determining the Limit of Detection in milk. The sensor's detection sensitivity was evaluated using different concentrations of E. coli cells. CIMs of E. coli containing 10⁶, 10⁵, 10⁴, and 10³ CFU/mL were prepared with each dilution being subjected to the cleavage test. Briefly, 30 μL of PFD liquid lubricant was applied to all surfaces, followed by submersion in 1×PBS buffer to remove excess PFD. Next, 10 μL of milk, 10 μL of the respective concentration of E. coli cells, and 20 μL of 2× reaction buffer were added to the samples, followed by incubation for 1 hour at room temperature (in a dark environment) to allow for cleavage to occur in the presence of bacteria. As before, 20 μL of milk and 20 μL of 2× Reaction Buffer were added to the surfaces of the samples containing no E. coli cells. Following a 1 hour incubation, the samples were imaged using a fluorescent microscope.

Fluorescent microscopy. All samples were imaged using a Zeiss inverted fluorescent microscope with an automatic bed (Zeiss Observer axio Z1). Images were obtained using the related Zen2 Blue Edition software. All images were obtained at a 5× surface magnification, with FAM and Texas Red light filters being used to capture the fluorescent images of the FAM- and TAMRA-labeled DNAs, respectively. The images were then analyzed using ImageJ software. The images were split into stacks with only the green or red stack being retained, depending on the fluorescent tag used. Finally, the brightness and contrast of the images were adjusted and all samples were given the same final parameters to ensure a fair comparison.

Results

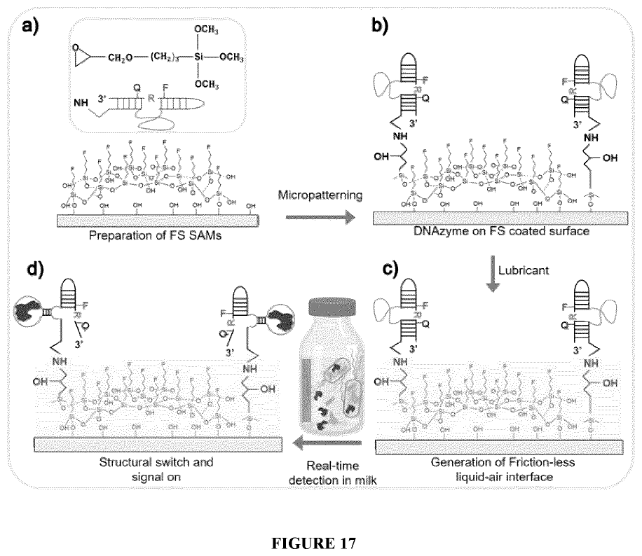

Making biofunctionalized surfaces with LIS and DNAzymes for detecting pathogens, such as bacteria, in complex fluids, such as milk, is illustrated in FIG. 17. Briefly, the surfaces were first treated with LIS, and then imaged to confirm the omniphobic-lubricant-infused coating's effect on the fluorescence signal, which would ultimately interfere with the selected imaging spectra for the DNAzyme. Next, amine-terminated DNAzyme probes were functionalized with epoxy silane (FIG. 17a) and immobilized onto the surface (FIG. 17b); they were then imaged to determine the immobilization efficiency. After the DNAzyme had been immobilized, an FDA approved lubricant was applied to the surfaces of the sensors to create a frictionless monolayer interface (FIG. 17c). The resultant sensor can then be installed inside food packaging, where it will release the quencher molecules and allow the fluorophores to exhibit the appropriate signal upon contacting bacteria (FIG. 17d).

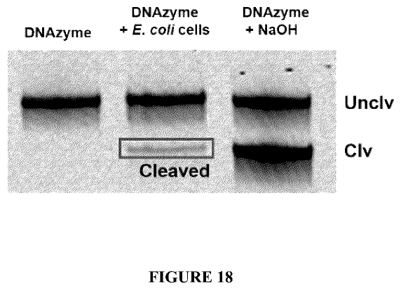

Since dairy products, including milk, contain the following fluorescent compounds: riboflavin, vitamin A, aromatic amino acids, maillard reaction products, porphyrins, chlorophylls, and lipid oxidation, milk samples used herein have a high green fluorescent background. Riboflavin, commonly known as Vitamin B2, has a maximum fluorescence at 520 nm, the same as FAM fluorescent dye. Therefore, to avoid combinatory signals due to the green autofluorescence from milk, DNAzyme sequences were designed with built-in TAMRA (carboxytetramethylrhodamine) dyes as the fluorophore probes. Consequently, the bright signal from the TAMRA-labeled DNA would indicate the successful hybridization of complementary strands. The emission spectrum of TAMRA ranges from 550 to 750 nm, with a peak emission at 615 nm. The lowest emission wavelength of this spectrum is 550 nm, higher than riboflavin; therefore, the spectrum of TAMRA precludes the detection of riboflavin. The cleavage activity of the TAMRA-labeled DNAzyme was evaluated using polyacrylamide gel electrophoresis. As shown in the gel image in FIG. 18, this modification does not inhibit the activity of the DNAzyme in the presence of E. coli. Therefore, the TAMRA-labeled DNAzyme was used herein to detect E. coli in milk. The complete DNAzyme sequence, named RFD-Ecl, and its component sequences are provided in Table 6.

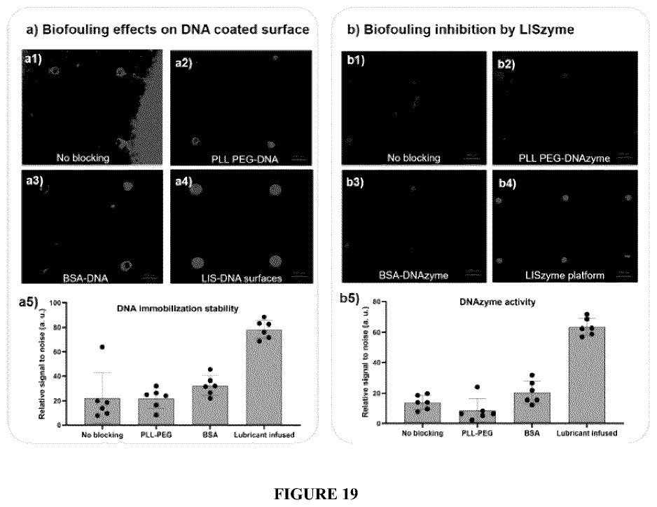

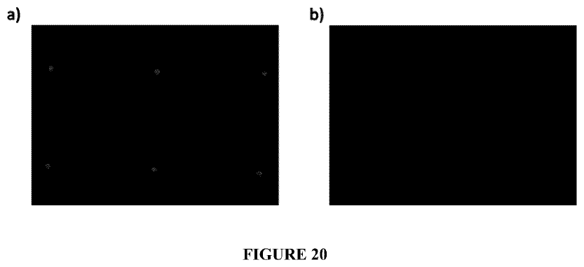

To study how the biofouling of milk impacts the performance of DNAzyme, a red dye labeled single-stranded DNA (named TRDNA) was first covalently micropatterned onto the surfaces and incubated in milk for 1 hour (FIG. 19a). FIG. 19(a1)-(a3) show that the gold standards in antibiofouling techniques were unable to overcome milk's adverse interaction with the sensor's surface. FIG. 19(a4) depicts DNA immobilization and fluorescence efficiency using LIS-DNAzyme surfaces, while FIG. 19(a5) shows the quantitative signal outcomes for each of the surfaces incubated in milk. FIG. 20 shows when DNAzyme is printed onto the surface without epoxy, there is no observable fluorescent signal after washing, indicating that DNAzyme cannot bind to the surface without epoxy.

Next, the effects of biofouling on the immobilization, functionality, and detection sensitivity of the DNAzyme sensors was examined. After immobilization, the sensors were exposed to milk spiked with E. coli cells (10⁶ CFU/mL) (FIG. 19b). The sensors were first exposed to the blocking agents, and then incubated as required. The sensors were then exposed to milk spiked with E. coli cells (10⁶ CFU/mL) and imaged after cleavage had occurred. Once again, an uncleaved DNAzyme sample (i.e., a sample that had been exposed to uncontaminated milk without any E. coli cells) was imaged to confirm the immobilization of the DNAzyme to the surface, and a cleaved DNAzyme without any blocking agent (FIG. 19(b1)) to determine the effectiveness of the blocking agents. The imaging results showed that the samples containing the three blocking agents (PLL-PEG, BSA, and perfluorodecalin (PFD) liquid lubricant) had brighter signals than the sample without any blocking agent. Furthermore, the results confirmed that the LIS-DNAzyme biosensor provided the best blocking, as it produced a significantly brighter signal (FIG. 19(b4)) compared to the others. In contrast, PLL-PEG was the lowest-performing of the three blocking agents, with very little signal and some background (FIG. 19(b2)), while BSA's signal brightness was comparable to that observed for LIS-DNAzymes (FIG. 19(b3)). These results were consistent with the preliminary results obtained using the TAMRA-labeled single-stranded DNA instead of the TAMRA-labeled DNAzyme (FIG. 19a). Overall, LIS-DNAzymes minimized biofouling the best with a signal-to-noise ratio of approximately 80 a.u., while all the other blocking agents had signal-to-noise ratios below 25 a.u. As a result, it was confirmed that the coupling of DNAzyme-immobilized sensors and LIS blockers is an effective approach for detecting bacteria in complex sample matrices such as milk.

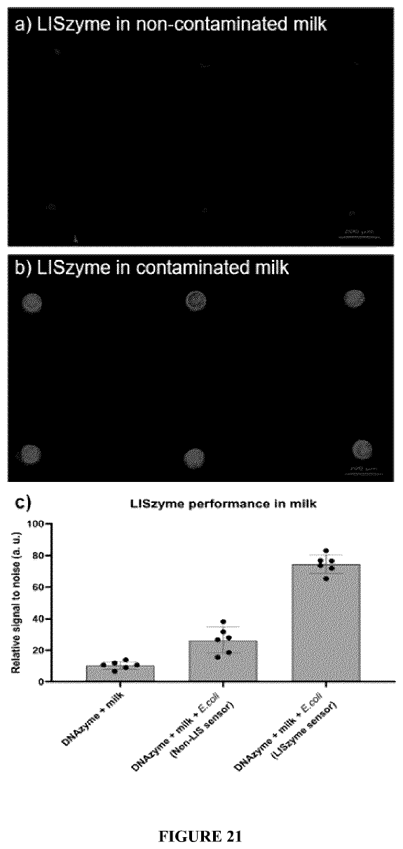

The performance of the LIS-DNAzyme biosensor was optimized as a function of probe concentration and mobility.. The results of the optimization process revealed that LIS-DNAzyme biosensors are capable of providing an eight-fold signal increase when detecting bacteria in milk, a marked improvement upon previous DNAzyme surface designs (FIG. 21). FIG. 21a shows that the uncleaved immobilized DNAzyme (with a fluorophore and a quencher dye) displayed a very low fluorescence signal, which indicates that it was successfully immobilized to the surface without losing its functionality. In contrast, FIG. 21b shows that the use of LIS to block the non-specific binding sites of the surface yielded a significantly higher signal-to-background ratio compared to the samples without lubricant. Overall, both sample sets (with and without lubricant) that were exposed to E. coli cells in milk had brighter signals than the uncleaved sample, indicating that the LIS treatment and the coupling process had no impact on the DNAzyme's functionality (FIG. 21c). The blocking agent, in accordance with the LIS treatment method, significantly increased the signal-to-background ratio, thus resulting in a brighter signal. This confirms the LIS treatment's effectiveness in increasing signal strength for complex fluid matrices.

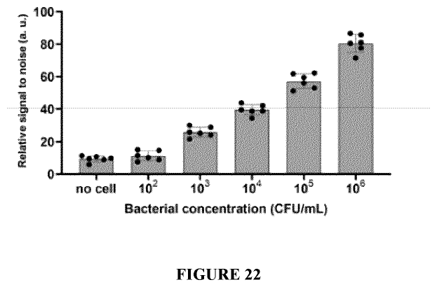

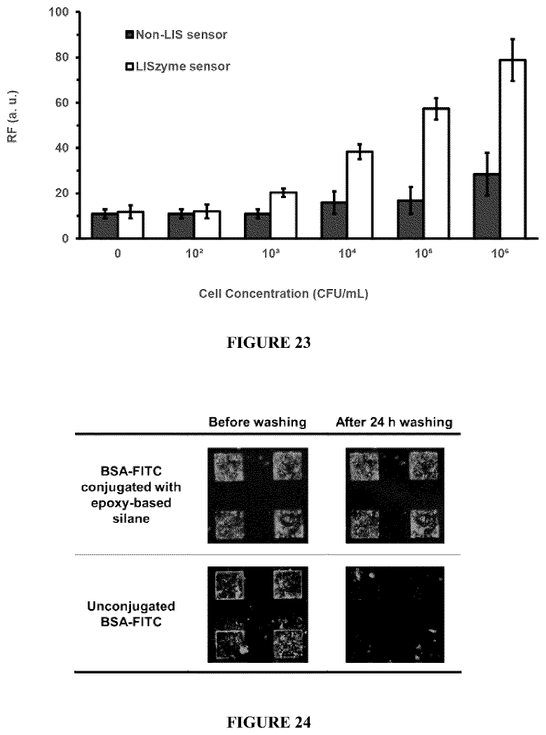

The limits of detection (LOD) for a similar DNAzyme reported in previous studies provided a gold standard for evaluating our LIS-DNAzyme biosesnsor's detection sensitivity in milk. Concentrations of E. coli cells ranging from 10⁶ to 10² CFU/mL were prepared. The LIS-DNAzymes sensors were then incubated in milk samples containing these different bacteria concentrations for one hour at room temperature in order to allow adequate cleavage of the DNAzyme. As the results in FIG. 22 show, the LOD is 1000 CFU/mL. Thus, the LIS-DNAzyme biosensor offers superior sensitivity for direct hands-free detection of bacteria in milk compared to those in previous studies and is promising in terms of commercial viability [91]. FIG. 23 demonstrates the dynamic range of the LIS-DNAzyme biosensor in comparison with the non-LIS treated sensors. As shown in FIG. 23, LIS treatment enhances the limits of detection of the platform by three-fold.

In summary, the developed LIS-DNAzyme biosensors provide unique capabilities for real-time hands-free detection of pathogens in complex food textures with an eight-fold signal increase for detecting E. coli in milk and significantly outperforming the currently available hands-free detection systems. Since the implemented lubricant (PFD) is approved by the FDA, the LIS-DNAzyme biosensors can be immobilized on food packaging and liquid bottles for real-time monitoring of target contaminations without the need to open the containers.

### Example 4. Modified Fluorosilane Treatment Parameters for Poly(Methyl Methacrylate) (PMMA) Surfaces

### Oxygen Plasma Treatment Time: About 15 Min;

Plasma treatment time was adjusted for PMMA surfaces in a way that the maximum amount of hydroxyl groups was obtained and to increase the hydrophilic properties of the surface. The contact angle of a pristine PMMA substrate is around 71°. After the plasma treatment, the contact angle drops to around 42°. Longer plasma treatment times do not change the amount of hydrophilicity anymore and thus, do not induce more hydroxyl groups. Shorter plasma treatment times (5 min and 10 min), however, cannot lower the contact angle very much, and does not provide sufficient amount of hydroxyls.

### CVD Treatment Time: About 30 Min (Using 200 μL of the Fluorosilane);

The conventional CVD treatment time for different substrates is around 3 hours. After the CVD time, the contact angle should increase to around 120°. But when the CVD treatment is done for half an hour, a contact angle of −109° is obtained. This confirms the presence of hydroxyls after the treatment. If the CVD is done for just 5 min, the same contact angle is achieved. Nevertheless, the slippery properties (i.e., sliding angle) of the surface changes. After half an hour FS CVD, the sliding angle of the PMMA surface (after adding the lubricant) was less than 5° which is the conventional sliding angle of a 3-hour FS treated surface. Therefore, the amount of FS groups after half an hour is sufficient to trap a thin layer of a fluorinated lubricant. When the FS treatment was done for shorter times, the sliding angle significantly increased. After 5 min FS treatment, for example, the sliding angle was more than 50°.

The coexistence of hydroxyls and FS groups after the modified FS treatment protocol was confirmed via XPS analysis. The results showed that the amount of hydroxyls, although decreased after FS CVD, was still higher than the initial amount of hydroxyls after plasma treatment (Table 1).

Moreover, if FS treatment is conducted on the surface for more than half an hour (1 hour or more), it may be harder to pattern the capture antibodies via the microcontact printing method using PDMS stamps (or potentially other contact printing methods). For instance, after an hour FS treatment, due to the high surface hydrophobicity, the capture antibody cannot be transferred from the PDMS stamp to the surface.

### Heat Treatment Time: About 30 Min at 90° C.;

Heat treatment above 90° C. deforms the PMMA substrates as the glass transition temperature of PMMA is not high. Heat treatment at less than 90° C. cannot promote the hydrolysis reactions needed for formation of self-assembled monolayers (SAM) of fluorosilane in a short time. Notably, the plasma induced hydroxyl groups on the surface of PMMA are only stable for a short time (around an hour). After an hour, the hydrophobic properties of PMMA are recovered. Thus, it is not recommended to perform the heat treatment step for more than half an hour since the hydroxyl groups can be removed from the surface.

The developed bioinks are prepared by mixing epoxy-based silane coupling agent, EDC/NHS, or other crosslinkers mentioned in the application, with the capture antibodies or DNAzymes (or other biomolecules of interest). Combination of this bioink with the modified FS treatment protocol enabled the creation of microarrays of biomolecules covalently bound to an FS treated surface, via either non-contact printing or contact printing approaches. This results in highly robust biosensors with excellent sensitivity and also provides an opportunity for multiplex detection. The robustness of the covalently patterned biomolecules was studied by comparing the stability of microcontact printed BSA-FITC (fluorescein isothiocyanate (FITC) conjugated bovine serum albumin (BSA)_on FS treated surfaces with and without using the bioink. In FIG. 24, BSA-FITC conjugated with GLYMO (glycidyloxypropyl)trimethoxysilane) was microcontact printed onto an FS treated PMMA substrate and the obtained fluorescent patterns were compared to a control sample where unconjugated BSA-FITC was patterned. The “before washing” images show higher yield of the protein obtained by use of GLYMO. The samples were then shaken for 24 hours in TBS Tween 20 (TBST) buffer to show the effect of conjugation in stability of the protein. In the unconjugated sample, the patterns were almost completely washed off due to the lack of covalent immobilization.

While the present application has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

### TABLES

TABLE 1
Quantification of the peak area percentages of C1s spectra for PMMA surfaces
before and after the surface modification. Data are shown as mean ± SD

| | Binding energy (eV) | | | | | |
|---|---|---|---|---|---|---|
| | 284.57 (±0.1) | 285.42 (±0.16) | 286.47 (±0.14) | 288.58 (±0.2) | 290.69 (±0.06) | 293.05 (±0.06) |
| | Chemical bond | | | | | |
| | C—C/C—H | C—C═O | C—O | O—C═O | —CF2 | —CF3 |
| | Peak area (%) | | | | | |
| Plain | 56.41 (±1.18) | 14.45 (±2.73) | 15.42 (±2.83) | 12.71 (±0.45) | 0 | 0 |
| PMMA | | | | | | |
| Plasma | 49.83 (±5.96) | 16.59 (±2.52) | 14.67 (±4.71) | 18.91 (±3.3) | 0 | 0 |
| treatment | | | | | | |
| FS | 23.58 (±1.06) | 23.48 (±4.3) | 6.97 (±1.62) | 14.19 (±0.98) | 26.25 (±3.44) | 5.52 (±0.64) |
| treatment | | | | | | |

TABLE 2
Standard curve MFI values and precision for the LIS IL-6 IFA
in buffer and plasma using two image processing methods

| | Buffer | | | | | | |
|---|---|---|---|---|---|---|---|
| | | | | | % Robust | Plasma | |
| | Background | % CV | Background | % CV | CV Background | Background | % CV |
| | Subtracted | Raw | Subtracted | Raw | Subtracted | Subtracted | Raw |
| [ ] | MFI FiJi | FiJi | MFI Chan-Vese | Chan-Vese | Chan-Vese | MFI FiJi | FiJi |
| pg/mL | ImageJ | ImageJ | Python | Python | Python | ImageJ | ImageJ |
| 2500 | 12278 | 14.0 | 12658 | 13.6 | 17.1 | 9576 | 15.0 |
| 312.5 | 8156 | 10.9 | 7938 | 10.9 | 23.8 | 5523 | 7.4 |
| 156 | 3499 | 10.1 | 3495 | 10.2 | 17.0 | 4291 | 9.5 |
| 40 | 2622 | 10.7 | 2737 | 10.8 | 31.2 | 3184 | 8.7 |
| 5 | 2310 | 8.1 | 2107 | 8.7 | 28.6 | 2439 | 4.4 |
| 1 | 1538 | 5.3 | 1465 | 5.7 | 32.2 | 1634 | 11.3 |
| 0.5 | 804 | 2.3 | 755 | 2.6 | 25.2 | 1207 | 3.5 |
| 0 | 150 | 1.4 | 19 | 1.3 | N/A | 338 | 3.0 |

TABLE 3
Two-sample T-test assuming unequal variances for buffer
LOD significance using Chan-Vese Python MFI values

| | 0 pg mL⁻¹ | 0.5 pg mL⁻¹ |
|---|---|---|
| Raw MFI | | |
| Mean | 11543 | 14000 |
| Observations | 18 | 18 |
| P(T <= t) two-tail | 4.06E−18 | |
| t Critical two-tail | 2.07 | |
| Background Subtracted MFI | | |
| Mean | 106 | 793 |
| Observations | 18 | 18 |
| P(T <= t) two-tail | 2.47E−10 | |
| t Critical two-tail | 2.05 | |

TABLE 4
Two-sample T-test assuming unequal variances for plasma
LOD significance using FiJi ImageJ processing

| | 0 pg mL⁻¹ | 0.5 pg mL⁻¹ |
|---|---|---|
| Raw MFI | | |
| Mean | 11350 | 14537 |
| Observations | 18 | 18 |
| P(T <= t) two-tail | 2.99E−20 | |
| t Critical two-tail | 2.04 | |
| Background Subtracted MFI | | |
| Mean | 338 | 1207 |
| Observations | 18 | 18 |
| P(T <= t) two-tail | 1.07E−06 | |
| t Critical two-tail | 2.04 | |

TABLE 5
Comparison of our developed IL-6 biosensor with previously reported strategies

| Ref. | Method | Advantages | Disadvantages | LOD^{a)} | Sample |
|---|---|---|---|---|---|
| T.-H. Chou, C.-Y. | Surface plasmon | The cell culture | Poor LOD and low | 1300 | Cell media |
| Chuang, C.-M. Wu, | resonance using a | medium could be | sensitivity | | |
| Cytokine 2010, 51, 107. | sandwich type | analyzed directly | | | |
| | immunoassay | without any prior | | | |
| | | procedures. | | | |
| R. Malhotra, V. Patel, J. | Electrochemical | High sensitivity and | Complexity and time- | 0.5 | Calf serum |
| P. Vaque, J. S. Gutkind, | immunosensor using | good reproducibility | consuming production, | | |
| J. F. Rusling, Anal. | CNTs | | no LOD was shown in | | |
| Chem. 2010, 82, 3118. | | | plasma | | |
| L. Luo, Z. Zhang, L. | Chemiluminescence | Good specificity and | Time consuming | 0.5 | Buffer |
| Hou, J. Wang, W. Tian, | immunoassay combining | high emission | assay, lack of good | | |
| Talanta 2007, 72, 1293. | ELISA and bis (2,4,6- | intensity | sensitivity in complex | | |
| | trichlorophenyl | | biofluids | | |
| | oxalate-hydrogen | | | | |
| | peroxide based CL | | | | |
| L. Luo, Z. Zhang, L. | Chemiluminescence | Improved linear | Complex design, low | 0.5 | Buffer |
| Hou, Anal. Chim. Acta | assay using gold | range and loading | sensitivity in complex | | |
| 2007, 584, 106. | nanoparticles | capability of the | fluids | | |
| | | assay, good | | | |
| | | sensitivity | | | |
| T. Li, M. Yang, Sensors | Electrochemical sensor | Good sensitivity and | Complex design, low | 1 | Buffer |
| Actuators B Chem. | using polyelectrolyte, | good reproducibility | sensitivity in complex | | |
| 2011, 158, 361. | ferrocene and CaCO₃ | | fluids, 100 pg/ml IL-6 | | |
| | nanoparticles | | in serum was the | | |
| | | | lowest concentration | | |
| | | | shown with good | | |
| | | | recovery | | |
| P. Chen, M. T. Chung, | Localized SPR-based | Rapid, capable of | No LOD was | 11.29 | Buffer |
| W. McHugh, R. Nidetz, | microfluidic optical | performing | assigned for biofluids | | |
| Y. Li, J. Fu, T. T. | biosensor | multiplex and low | | | |
| Cornell, T. P. Shanley, | | volume assays. | | | |
| K. Kurabayashi, ACS | | | | | |
| Nano 2015, 9, 4173. | | | | | |
| X. Hun, Z. Zhang, | Immunofluorescence | Simplicity and | Low sensitivity, use | 7 | Buffer |
| Biosens. Bioelectron. | assay using core-shell | reproducibility | of beads | | |
| 2007, 22, 2743. | nanoparticles | | | | |
| J. Wu, Y. Chen, M. | Microfluidic | Good sensitivity and | Incapable of detecting | 1 | Buffer |
| Yang, Y. Wang, C. | immunoassay combined | linear dynamic | IL-6 in complex | | |
| Zhang, M. Yang, J. Sun, | with the streptavidin- | range, good | biofluids (e.g. | | |
| M. Xie, X. Jiang, Anal. | biotin-peroxidase (SA- | selectivity | plasma) | | |
| Chim. Acta 2017, 982, | B-HRP) nanocomplex- | | | | |
| 138. | signal amplification | | | | |
| | system | | | | |
| H. Chen, T. K. Choo, J. | Electronic detection | Good stability and | Incapable of detecting | 1.37 | Buffer |
| Huang, Y. Wang, Y. | using liquid-gated field- | sensitivity | IL-6 in unprocessed | | |
| Liu, M. Platt, A. | effect transistor (FET) | | biofluids. Detection | | |
| Palaniappan, B. | sensors and CNTs | | only showed in 100 | | |
| Liedberg, A. I. Y. Tok, | | | times diluted serum | | |
| Mater. Des. 2016, 90, | | | | | |
| 852. | | | | | |
| G. Liu, K. Zhang, A. | Sandwich-based IFA | Requires low sample | Complex design, | 1 | Buffer |
| Nadort, M. R. | using gold nanoparticle | volume, capable of | incapable of detecting | | |
| Hutchinson, E. M. | modified silica optical | spatially localized | IL-6 in complex | | |
| Goldys, ACS Sensors | fibers | cytokine detection | fluids | | |
| 2017, 2, 218. | | | | | |
| G. Wang, H. Huang, B. | Electrochemical | Very high sensitivity | Complex design, | 0.05 | Buffer |
| Wang, X. Zhang, L. | immunosensor using | and low LOD in | Lack of sensitivity in | | |
| Wang, Chem. Commun. | supersandwich | buffer | complex biofluids | | |
| 2012, 48, 720. | multienzyme-DNA | | | | |
| | label | | | | |
| X. P. Liu, X. L. Xie, Y. | Photoelectrochemical | Very low LOD in | Lack of sensitivity in | 0.033 | Buffer |
| P. Wei, C. jie Mao, J. S. | sensing using LaFeO₃ | buffer | complex biofluids. | | |
| Chen, H. L. Niu, J. M. | NPs deposited on FTO | | 100 pg/ml in serum | | |
| Song, B. K. Jin, | glass | | was the lowest | | |
| Microchim. Acta 2018, | | | detection trial done in | | |
| 185, 52. | | | the paper. | | |
| M. Tertiş, B. Ciui, M. | Electrochemical | High sensitivity in | The device | 0.33 | Buffer |
| Suciu, R. Săndulescu, C. | aptasensor using a | buffer, high | functionality in | | |
| Cristea, Electrochim. | composite of | analytical range | unprocessed biofluids | | |
| Acta 2017, 258, 1208. | polypyrrole and gold | | is not shown | | |
| | nanoparticles | | | | |
| J. Sabaté del Río, O. Y. | Electrochemical sensor | Good sensitivity and | Complex design | 23 | Plasma |
| F. Henry, P. Jolly, D. E. | using porous composite | stability in complex | | | |
| Ingber, Nat. Nanotechnol. | of BSA, AuNWs, and | liquids | | | |
| 2019, 14, 1143. | CNTs or AuNPs | | | | |
| The present | LIS-IFA using patterned | Low cost and simple | Relatively longer | 0.5 | Plasma and |
| disclosure | capture antibodies on a | design, low LOD in | time of detection due | | Buffer |
| | functional lubricant | complex biofluids, | to using well plates. | | |
| | infused surface | ability to multiplex | Can be improved in | | |
| | | detection for high- | the future using | | |
| | | throughput applications | microfluidic based | | |
| | | | assays | | |

^{a)}LOD is in pg mL⁻¹

TABLE 6
The sequences of and modifications to all
oligonucleotides used herein.

| SEQ | | | |
|---|---|---|---|
| ID | | | |
| NO. | Name | Sequence | Description |
| 1 | NH- | 5′-NH₂TTTTTCACGG ATCCTGACAA | Amine-labeled |
| | EC1 | GGATGTGGTT GTCGAGACCT GCGACCGGAA | DNAzyme |
| | | CACTACACTG TGTGGGATGG ATTTCTTTAC | sequence for |
| | | AGTTGTGTGCA GCTCCGTCCG-3′ | E. coli K12 |
| 2 | TS1 | 5′-ACTCTTCCTA GCTrAQGGTT | Fluorogenic |
| | | CGATCAAGA-3′ | substrate |
| | | (T: TAMRA-dT; | |
| | | rA: riboadenosine; | |
| | | Q: BHQ2-dT) | |
| 3 | LT | 5′-CTAGGAAGAGTCGGACGGAGCTG-3′ | Ligation template |
| | | | for ligating NH- |
| | | | EC1 to TS1 |
| 4 | RFD- | 5′-NH₂TTTTTCACGG ATCCTGACAA | Complete |
| | EC1 | GGATGTGGTT GTCGAGACCT GCGACCGGAA | DNAzyme |
| | | CACTACACTG TGTGGGATGG ATTTCTTTAC | sequence |
| | | AGTTGTGTGC AGCTCCGTCC GACTCTTCC | including NH-EC1 |
| | | TAGCTrAQGGT | and TS1 |
| | | TCGATCAAGA-3′ | |
| 5 | TRDNA | 5′-/5AmMC12/TTTTTCACGG ATCCTGACAA | Texas red-labeled |
| | | GGAT/3TEX615/ | ssDNA sequence |
| 6 | FAMDNA | 5′-/5AmMC12/TTTTTCACGG ATCCTGACAA | FAM-labeled |
| | | GGAT/3TFAM/ | ssDNA sequence |

### REFERENCES

1) B. Kasemo, Biological surface science, Surf. Sci. 500 (2002) 656-677. doi:https://doi.org/10.1016/S0039-6028(01)01809-X.
2) M. Van de Voorde, M. Werner, H. Fecht, The Nano-Micro Interface: Bridging the Micro and Nano Worlds, (2015).
3) E. Sackmann, M. Tanaka, Supported membranes on soft polymer cushions: fabrication, characterization and applications, Trends Biotechnol. 18 (2000) 58-64. doi:https://doi.org/10.1016/S0167-7799(99)01412-2.
4) F. Frederix, K. Bonroy, W. Laureyn, G. Reekmans, A. Campitelli, W. Dehaen, G. Maes, Enhanced performance of an affinity biosensor interface based on mixed self-assembled monolayers of thiols on gold, Langmuir. 19 (2003) 4351-4357. doi:10.1021/1a026908f.
5) N. Tort, J.-P. Salvador, M.-P. Marco, Multimodal plasmonic biosensing nanostructures prepared by DNA-directed immobilization of multifunctional DNA-gold nanoparticles, Biosens. Bioelectron. 90 (2017) 13-22. doi:https://doi.org/10.1016/j.bios.2016.11.022.
6) D. Zheng, K. G. Neoh, E.-T. Kang, Bifunctional coating based on carboxymethyl chitosan with stable conjugated alkaline phosphatase for inhibiting bacterial adhesion and promoting osteogenic differentiation on titanium, Appl. Surf. Sci. 360 (2016) 86-97. doi:https://doi.org/10.1016/j.apsusc.2015.11.003.
7) M. Zychowicz, D. Dziedzicka, D. Mehn, H. Kozlowska, A. Kinsner-Ovaskainen, P. P. Stepień, F. Rossi, L. Buzanska, Developmental stage dependent neural stem cells sensitivity to methylmercury chloride on different biofunctional surfaces, Toxicol. Vitr. 28 (2014) 76-87. doi:https://doi.org/10.1016/j.tiv.2013.06.023.
8) D. Roe, B. Karandikar, N. Bonn-Savage, B. Gibbins, J.-B. Roullet, Antimicrobial surface functionalization of plastic catheters by silver nanoparticles, J. Antimicrob. Chemother. 61 (2008) 869-876. http://dx.doi.org/10.1093/jac/dkn034.
9) D. Shenoy, W. Fu, J. Li, C. Crasto, G. Jones, C. DiMarzio, S. Sridhar, M. Amiji, Surface functionalization of gold nanoparticles using hetero-bifunctional poly(ethylene glycol) spacer for intracellular tracking and delivery, Int. J. Nanomedicine. 1 (2006) 51-57. doi:10.2147/nano.2006.1.1.51.
10) A. Fujishima, X. Zhang, D. A. Tryk, TiO2 photocatalysis and related surface phenomena, Surf. Sci. Rep. 63 (2008) 515-582. doi:10.1016/j.surfrep.2008.10.001.
11) S. Grivennikov, M. Karin, Cancer Cell 2008, 13, 7.
12) Y.-C. Chung, Y.-F. Chang, J. Surg. Oncol. 2003, 83, 222.
13) Z. Culig, M. Puhr, Mol. Cell. Endocrinol. 2012, 360, 52.
14) C. Dethlefsen, G. Hojfeldt, Breast Cancer Res. Treat. 2013, 138, 657.
15) H. Yanagawa, S. Sone, Y. Takahashi, T. Haku, S. Yano, T. Shinohara, T. Ogura, Br. J. Cancer 1995, 71, 1095.
16) M. Lesina, M. U. Kurkowski, K. Ludes, S. Rose-john, M. Treiber, G. Kloppel, A. Yoshimura, W. Reindl, B. Sipos, S. Akira, R. M. Schmid, H. Algul, Cancer Cell 2011, 19, 456.
17) N. Kumari, B. S. Dwarakanath, A. Das, A. N. Bhatt, Tumor Biol. 2016, 37, 11553.
18) T. Tanaka, M. Narazaki, T. Kishimoto, Cold Spring Harb Perspect Biol 2014, 6, a016295.
19) T. Herold, V. Jurinovic, C. Amreich, J. C. Hellmuth, M. von Bergwelt-Baildon, M. Klein, T. Weinberger, medRxiv 2020, 2020.04.01.20047381.
20) X. Chen, B. Zhao, Y. Qu, Y. Chen, J. Xiong, Y. Feng, D. Men, Q. Huang, Y. Liu, B. Yang, J. Ding, F. Li, Clin. Infect. Dis. 2020, ciaa449.
21) F. Khosravi, S. M. Loeian, B. Panchapakesan, Biosensors 2017, 7, 17.
22) M. Helle, L. Boeije, E. de Groot, A. de Vos, L. Aarden, J. Immunol. Methods 1991, 138, 47.
23) J. Sabaté del Rio, O. Y. F. Henry, P. Jolly, D. E. Ingber, Nat. Nanotechnol. 2019, 14, 1143.
24) B. S. Munge, C. E. Krause, R. Malhotra, V. Patel, J. Silvio Gutkind, J. F. Rusling, Electrochem. commun. 2009, 11, 1009.
25) T. Li, M. Yang, Sensors Actuators B Chem. 2011, 158, 361.
26) R. Malhotra, V. Patel, J. P. Vague, J. S. Gutkind, J. F. Rusling, J. P. Vague, J. S. Gutkind, J. F. Rusling, Anal. Chem. 2010, 82, 3118.
27) M. A. Khan, M. Mujahid, Sensors (Switzerland) 2020, 20, 646.
28) G. Wang, H. Huang, B. Wang, X. Zhang, L. Wang, Chem. Commun. 2012, 48, 720.
29) M. Tertis, B. Ciui, M. Suciu, R. Sändulescu, C. Cristea, Electrochim. Acta 2017, 258, 1208.
30) C. Diacci, M. Berto, M. Di Lauro, E. Bianchini, M. Pinti, D. Simon, F. Biscarini, C. A. Bortolotti, Biointerphases 2017, 12, 10.1116.
31) N. Liu, H. Yi, Y. Lin, H. Zheng, X. Zheng, D. Lin, H. Dai, Microchim. Acta 2018, 185, 277.
32) X. P. Liu, X. L. Xie, Y. P. Wei, C. jie Mao, J. S. Chen, H. L. Niu, J. M. Song, B. K. Jin, Microchim. Acta 2018, 185, 52.
33) T.-H. Chou, C.-Y. Chuang, C.-M. Wu, Cytokine 2010, 51, 107.
34) J. F. Masson, ACS Sensors 2017, 2, 16.
35) P. Chen, M. T. Chung, W. McHugh, R. Nidetz, Y. Li, J. Fu, T. T. Cornell, T. P. Shanley, K. Kurabayashi, ACS Nano 2015, 9, 4173.
36) L. Luo, Z. Zhang, L. Hou, Anal. Chim. Acta 2007, 584, 106.
37) L. Luo, Z. Zhang, L. Hou, J. Wang, W. Tian, Talanta 2007, 72, 1293.
38) J. Wu, Y. Chen, M. Yang, Y. Wang, C. Zhang, M. Yang, J. Sun, M. Xie, X. Jiang, Anal. Chim. Acta 2017, 982, 138.
39) J. Deng, M. Yang, J. Wu, W. Zhang, X. Jiang, Anal. Chem. 2018, 90, 9132.
40) G. Liu, M. Qi, M. R. Hutchinson, G. Yang, E. M. Goldys, Biosens. Bioelectron. 2016, 79, 810.
41) J. F. Djoba Siawaya, T. Roberts, C. Babb, G. Black, H. J. Golakai, K. Stanley, N. B. Bapela, E. Hoal, S. Panda, P. van Heiden, G. Walzl, PLoS One 2008, 3, e2535.
42) G. Liu, K. Zhang, A. Nadort, M. R. Hutchinson, E. M. Goldys, ACS Sensors 2017, 2, 218.
43) X. Hun, Z. Zhang, Biosens. Bioelectron. 2007, 22, 2743.
44) P. D′Orazio, Clin. Chim. Acta 2011, 412, 1749.
45) R. E. Saunders, B. Derby, Inkjet printing biomaterials for tissue engineering:
bioprinting, Int. Mater. Rev. 59 (2014) 430-448. doi:10.1179/1743280414Y.0000000040.
46) D. J. Graber, T. J. Zieziulewicz, D. A. Lawrence, W. Shain, J. N. Turner, Antigen binding specificity of antibodies patterned by microcontact printing, Langmuir. 19 (2003) 5431-5434. doi:10.1021/1a034199f.
47) Y. Temiz, R. D. Lovchik, E. Delamarche, Capillary-Driven Microfluidic Chips for Miniaturized Immunoassays: Patterning Capture Antibodies Using Microcontact Printing and Dry-Film Resists BT—Microchip Diagnostics: Methods and Protocols, in: V. Taly, J.-L. Viovy, S. Descroix (Eds.), Springer New York, New York, NY, 2017: pp. 37-47. doi:10.1007/978-1-4939-6734-6_3.
48) S. Sathish, S. G. Ricoult, K. Toda-Peters, A. Q. Shen, Microcontact printing with aminosilanes: creating biomolecule micro- and nanoarrays for multiplexed microfluidic bioassays, Analyst. 142 (2017) 1772-1781. doi:10.1039/C7AN00273D.
49) W. J. Yang, T. Cai, K. G. Neoh, E. T. Kang, S. L. M. Teo, D. Rittschof, Barnacle cement as surface anchor for “clicking” of antifouling and antimicrobial polymer brushes on stainless steel, Biomacromolecules. 14 (2013) 2041-2051. doi:10.1021/bm400382e.
50) C. Rodriguez-Emmenegger, M. Houska, A. B. Alles, E. Brynda, Surfaces Resistant to Fouling from Biological Fluids: Towards Bioactive Surfaces for Real Applications, Macromol. Biosci. 12 (2012) 1413-1422. doi:10.1002/mabi.201200171.
51) J. Jiang, L. Zhu, L. Zhu, H. Zhang, B. Zhu, Y. Xu, Antifouling and antimicrobial polymer membranes based on bioinspired polydopamine and strong hydrogen-bonded poly(n-vinyl pyrrolidone), ACS Appl. Mater. Interfaces. 5 (2013) 12895-12904. doi:10.1021/am403405c.
52) A. K. Epstein, T.-S. Wong, R. A. Belisle, E. M. Boggs, J. Aizenberg, Liquid-infused structured surfaces with exceptional anti-biofouling performance, Proc. Natl. Acad. Sci. U.S.A 109 (2012) 13182-13187. doi:10.1073/pnas.1201973109.
53) M. Tanaka, E. Sackmann, Supported membranes as biofunctional interfaces and smart biosensor platforms, Phys. Status Solidi Appl. Mater. Sci. 203 (2006) 3452-3462. doi:10.1002/pssa.200622464.
54) M. A. Sentandreu, L. Aubry, F. Toldrá, A. Ouali, Blocking agents for ELISA quantification of compounds coming from bovine muscle crude extracts, Eur. Food Res. Technol. 224 (2007) 623-628. doi:10.1007/s00217-006-0348-3.
55) J. W. Haycock, Polyvinylpyrrolidone as a blocking agents in immunochemical studies, Anal Biochem. 208 (1993) 397-399. doi:10.1006/abio.1993.1068.
56) T.-S. Wong, S. H. Kang, S. K. Y. Tang, E. J. Smythe, B. D. Hatton, A. Grinthal, J. Aizenberg, Bioinspired self-repairing slippery surfaces with pressure-stable omniphobicity, Nature. 477 (2011) 443-447. doi:10.1038/nature10447.
57) M. Villegas, Y. Zhang, N. Abu Jarad, L. Soleymani, T. F. Didar, ACS Nano 2019, 13, 8517.
58) M. Badv, S. M. Imani, J. I. Weitz, T. F. Didar, ACS Nano 2018, 12, 10890-10902.
59) A. Hosseini, M. Villegas, J. Yang, M. Badv, I. W. Jeffrey, L. Soleymani, F. T. Didar, Adv. Mater. Interfaces 2018, 5, 1800617.
60) M. Badv, I. H. Jaffer, J. I. Weitz, T. F. Didar, Sci. Rep. 2017, 7, 11639.
61) N. Vogel, R. A. Belisle, B. Hatton, T. S. Wong, J. Aizenberg, Nat. Commun. 2013, 4, 1.
62) D. C. Leslie, A. Waterhouse, J. B. Berthet, T. M. Valentin, A. L. Watters, A. Jain, P. Kim, B. D. Hatton, A. Nedder, K. Donovan, E. H. Super, C. Howell, C. P. Johnson, T. L. Vu, D. E. Bolgen, S. Rifai, A. R. Hansen, M. Aizenberg, M. Super, J. Aizenberg, D. E. Ingber, Donald E Ingber, Nat. Biotechnol. 2014, 32, 1134.
63) J. Li, T. Kleintschek, A. Rieder, Y. Cheng, T. Baumbach, U. Obst, T. Schwartz, P. A. Levkin, ACS Appl. Mater. Interfaces 2013, 5, 6704.
64) P. Wang, Z. Lu, D. Zhang, Corros. Sci. 2015, 93, 159.
65) S. Yuan, S. Luan, S. Yan, H. Shi, J. Yin, ACS Appl. Mater. Interfaces 2015, 7, 19466.
66) S. Sathish, N. Ishizu, A. Q. Shen, ACS Appl. Mater. Interfaces 2019, 11, 46350.
67) A. M. D. Wan, D. Devadas, E. W. K. Young, Sensors Actuators B Chem. 2017, 253, 738.
68) G. Beamson, D. Briggs, J. Chem. Educ. 1993, 70, A25.
69) S. Pletincx, K. Marcoen, L. Trotochaud, L. L. Fockaert, J. M. C. Mol, A. R. Head, O. Karslioglu, H. Bluhm, H. Terryn, T. Hauffman, Sci. Rep. 2017, 7, 13341.
70) V. Dugas, Y. Chevalier, J. Colloid Interface Sci. 2003, 264, 354.
71) A. Shaken, S. Rahmani, S. M. Imani, M. Osborne, H. Yousefi, T. F. Didar, in Woodhead Publ. Ser. Electron. Opt. Mater. (Eds.: K. Pal, H.-B. Kraatz, A. Khasnobish, S. Bag, I. Banerjee, U. Kuruganti), Woodhead Publishing, 2019, pp. 635-699.
72) X. Cui, Y. Gao, S. Zhong, Z. Zheng, Y. Cheng, H. Wang, J. Polym. Res. 2012, 19, DOI 10.1007/s10965-012-9832-6.
73) T. Song, Q. Liu, J. Liu, W. Yang, R. Chen, X. Jing, K. Takahashi, J. Wang, Appl. Surf. Sci. 2015, 355, 495.
74) C. S. Park, E. Y. Jung, H. J. Jang, G. T. Bae, B. J. Shin, H. S. Tae, Polymers (Basel). 2019, 11, DOI 10.3390/polym11030396.
75) H. R. Tantawy, B. A. F. Kengne, D. N. Mcllroy, T. Nguyen, D. Heo, Y. Qiang, D. E. Aston, J. Appl. Phys. 2015, 118, DOI 10.1063/1.4934851.
76) L. Zhang, Y. Li, L. Zhang, D. W. Li, D. Karpuzov, Y. T. Long, Int. J. Electrochem. Sci. 2011, 6, 819.
77) A. Shukla, S. D. Bhat, V. K. Pillai, J. Memb. Sci. 2016, 520, 657.
78) G. Panomsuwan, N. Saito, T. Ishizaki, J. Mater. Chem. A 2015, 3, 9972.
79) A. R. Barron, Physical Methods in Chemistry and Nano Science, Rice University, Houston, Texas, 2012.
80) M. Badv, J. I. Weitz, T. F. Didar, Small 2019, 15, 1905562.
81) M. Badv, C. Alonso-Cantu, A. Shaken, Z. Hosseinidoust, J. I. Weitz, T. F. Didar, ACS Biomater. Sci. Eng. 2019, 5, 6485.
82) J. Schindelin, I. Arganda-Carreras, E. Frise, V. Kaynig, M. Longair, T. Pietzsch, S. Preibisch, C. Rueden, S. Saalfeld, B. Schmid, J.-Y. Tinevez, D. J. White, V. Hartenstein, K. Eliceiri, P. Tomancak, A. Cardona, Nat. Methods 2012, 9, 676.
83) P. Getreuer, Image Process. Line 2012, 2, 214.
84) S. Van Der Walt, J. L. Schonberger, J. Nunez-Iglesias, F. Boulogne, J. D. Warner, N. Yager, E. Gouillart, T. Yu, PeerJ 2014, 2, e453.
85) L. Fayad, M. J. Keating, J. M. Reuben, S. O'Brien, B. N. Lee, S. Lerner, R. Kurzrock, Blood 2001, 97, 256.
86) H. Knüpfer, R. Preiß, Breast Cancer Res. Treat. 2007, 102, 129.
87) J. Nakashima, M. Tachibana, Y. Horiguchi, M. Oya, T. Ohigashi, H. Asakura, M. Murai, Clin. Cancer Res. 2000, 6, 2702 LP.
88) D. J. George, S. Halabi, T. F. Shepard, B. Sanford, N. J. Vogelzang, E. J. Small, P. W. Kantoff, Clin. Cancer Res. 2005, 11, 1815 LP.
89) M. Plante, S. C. Rubin, G. Y. Wong, M. G. Federici, C. L. Finstad, G. A. Gastl, Cancer 1994, 73, 1882.
90) J. Kim, A. S. Campbell, B. E.-F. de Ávila, J. Wang, Nat. Biotechnol. 2019, 37, 389.
91) A. Mortari, L. Lorenzelli. Biosens. Bioelectron. 2014, 60, 8.

1. A method for fabricating a biofunctionalized surface on a substrate, wherein the substrate comprises hydroxyl groups on the surface to be biofunctionalized, the method comprising:
(a) covalently attaching organosilane groups to less than all of the hydroxyl groups on the surface of the substrate;
(b) covalently attaching one or more biospecies to the surface of the substrate; and
(c) applying a lubricant to the substrate,
wherein the biospecies comprises a biorecognition element that detects a target analyte in a sample.

2. The method of claim 1, wherein the organosilane groups are attached to less than all of the hydroxyl groups on the surface of the substrate in (a) by contacting the substrate with an organosilanating reagent for about 5 minutes to about 30 minutes at a temperature of about 20° C. to about 90° C. to provide unmodified hydroxyl groups and modified hydroxyl groups and the biospecies is covalently attached in (b) to the unmodified hydroxyl groups.

3. The method of claim 1, wherein the organosilane groups are attached to less than all of the hydroxyl groups on the surface of the substrate in (a) by first treating the substrate with CO₂ plasma under conditions to convert only a portion of the hydroxyl groups to carboxyl groups and covalently attaching organosilane groups to the unconverted hydroxyl groups, and the biospecies is covalently attached in (b) to the carboxyl groups.

4. The method of claim 1, wherein covalently attaching organosilane groups comprises chemical vapor deposition or liquid phase deposition.

5. The method of claim 1, wherein covalently attaching the biospecies comprises applying a covalent crosslinking agent to the substrate before applying the biospecies to the substrate, or combining a covalent crosslinking agent with the biospecies into a mixture then applying the mixture to the substrate.

6. (canceled)

7. The method of claim 1, wherein covalently attaching the biospecies comprises positioning the biospecies in a distinct pattern on the surface.

8. The method of claim 1, wherein covalently attaching the biospecies comprises non-contact printing, optionally inkjet printing and/or spraying.

9. The method of claim 1, wherein covalently attaching the biospecies comprises contact printing, optionally microcontact printing, roll-to-roll printing and/or stamping.

10. The method of claim 1, wherein the substrate comprises a metallic, polymeric and/or glass material, optionally a nanoparticle.

11. The method of claim 1, wherein the organaosilane is a fluorosilane.

12. The method of claim 11, wherein the fluorosilane comprises 1H,1H,2H,2H-perfluorooctyltriethoxysilane, trichloro(1H,1H,2H,2H-perfluorooctyl)silane, heptadecafluoro-1,1,2,2-tetrahydrodecyl trichlorosilane and/or 1H,1H,2H,2H-perfluorodecyltrimethoxysilane.

13. The method of claim 1, wherein organosilane groups comprises n-propyltrichlorosilane, and/or methyltrichlorosilane.

14. The method of claim 1, further comprising micro- or nano-sized structures on the surface, and/or wherein the biospecies are positioned in a distinct pattern on the surface.

15. The method of claim 1, wherein the lubricant comprises a perfluorotrialkylamine, a perfluoroalkylether or perfluoroalkylpolyether, a perfluoroalkane, a perfluorocycloalkane, perfluoroperhydrophenanthrene (PFPP) and/or a perfluorohaloalkane.

16. The method of claim 5, wherein the covalent crosslinking agent comprises a silane coupling agent comprising a mono-, di- or tri-functional silane.

17. (canceled)

18. The method of claim 16, wherein the silane coupling agent is selected from (3-aminopropyl)triethoxysilane (APTES), (3-aminopropyl)trimethoxysilane (APTMS), 3-mercaptopropyl trimethoxysilane (MPTMS) and/or glycidyloxypropyl)trimethoxysilane (GLYMO).

19. The method of claim 5, wherein the covalent crosslinking agent comprises a carbodiimide crosslinker, glutaraldehyde, glycidyl methacrylate, hexamethylenediamine (NMDA), 1,3-diaminopropane (DAP), N-lithioethylenediamine, N-lithiodiaminopropane, an epoxy group and/or succinimide ester such as n-γ-maleimidobutyryl-oxysuccinimide ester, or wherein the covalent crosslinking agent comprises a polymer, optionally in combination with a silane.

20. (canceled)

21. The method of claim 20, wherein the polymer comprises cyclophane-containing polymers, poly(allylamine hydrochloride), poly(ethyleneimine), poly(acrylic acid), functional polyethylene glycol (PEG) (e.g. NHS-PEG), amine functional polyacrylamide, poly(ethyleneimine) (PEI), poly(allylamine hydrochloride) (PAH), and, polyallylamine, amine functional parylenes, and/or hyperbranched polyglycerol.

22. The method of claim 1, wherein the biospecies comprises a biomolecule, virus, cell and/or tissue, or wherein the biomolecule comprises a protein, peptide and/or nucleic acid, for example wherein the biomolecule is an antibody or a DNAzyme.

23.-26. (canceled)

27. A biosensor comprising a biofunctionalized surface prepared using a method of claim 1, wherein the biofunctionalized surface is capable of preventing non-specific adsorption and/or wherein the biosensor provides and multiplex detection of different target analytes.

28.-33. (canceled)
